(19) United States
(12) Patent Application Publication
(10) Pub. No.: US 2017/0304350 A1
(43) Pub. Date: Oct. 26, 2017

(54) PHARMACEUTICAL COMPOSITION FOR TREATING A VIRAL INFECTION

(71) Applicant: London School of Hygiene & Tropical Medicine, London (GB)

(72) Inventor: Polly Roy, London (GB)

(73) Assignee: London School of Hygiene & Tropical Medicine, London (GB)

(21) Appl. No.: 15/518,120

(22) PCT Filed: Oct. 7, 2015
(86) PCT No.: PCT/GB2015/052925

§ 371 Date: Apr. 10, 2017

(30) Foreign Application Priority Data
Oct. 14, 2014 (GB) 1418135.8

(51) Int. Cl. A61K 31/712; A61K 45/06; C12N 15/115; C12N 15/113

(52) U.S. Cl. CPC A61K 31/712; C12N 15/1131; A61K 45/06; C12N 15/115; A61K 2300/00; C12N 2720/12011

(57) ABSTRACT

The invention concerns a pharmaceutical composition for treating a viral infection caused by a member of the Reoviridae family; a method of treatment involving the use of same and use of the anti-viral to treat said viral infection. The agent has use in both humans and animals.

### FIELD OF THE INVENTION

The invention concerns a pharmaceutical composition for treating a viral infection caused by a member of the Reoviridae family; a method of treatment involving the use of same; and use of the anti-viral to treat said viral infection. The invention has use in both humans and animals.

### BACKGROUND OF THE INVENTION

How virus genomes, particularly those in the Reoviridae, are packaged into their protective coats, or capsids, during infection is unknown. This is especially so for viruses with multipartite genomes, as a copy of each segment of the genome must be incorporated into the capsid for the virus to be viable.

Members of the Reoviridae family can affect the gastrointestinal system (such as Rotavirus) and respiratory tract. Viruses in the family Reoviridae have genomes consisting of segmented, double-stranded RNA (dsRNA).

Reoviruses are non-enveloped and have an icosahedral capsid (T-13) composed of an outer and inner protein shell. The genomes of viruses in Reoviridae contain 10-12 segments which are grouped into three categories corresponding to their size: L (large), M (medium) and S (small). Typically segments range from about 3.9 to 1 kbp and each segment encodes 1-3 proteins.

Rotavirus is a genus of double-stranded RNA virus in the family Reoviridae. There are eight species of this virus, referred to as A, B, C, D, E, F, G and H. Rotavirus A, the most common species, causes more than 90% of rotavirus infections in humans. The genome of rotavirus is segmented and consists of 11 unique double helix molecules of RNA which are 18,555 nucleotides in total. The RNA is surrounded by a three-layered icosahedral non-enveloped protein capsid. There are six viral proteins (VPs) that form the virus particle (virion). These structural proteins are called VP1, VP2, VP3, VP4, VP6 and VP7. In addition to the VPs, there are six non-structural proteins (NSPs), that are only produced in cells infected by rotavirus. These are called NSP1, NSP2, NSP3, NSP4, NSP5 and NSP6. Each helix, or segment, is a gene numbered 1 to 11 by decreasing size.

Bluetongue virus (BTV) is a complex, multi-layered, segmented double-stranded RNA virus and is the type member of the Orbiviruses, a genus in the family Reoviridae. As such it shares a virus family relationship with several other scientifically and medically important viruses (e.g. Rotaviruses). BTV is transmitted by insect vectors, replicating in both insect and mammalian cells, and can cause high morbidity and mortality in animals. BTV particles are non-enveloped, architecturally complex particles organised in two capsids. The outer capsid is composed of VP2 and VP5, which are responsible for virus entry in mammalian cells. The icosahedral inner capsid or core, with a diameter of 75 nm, is composed of two protein layers, the surface layer of 260 trimers of VP7 (38 kDa) which is built on a thin scaffold made up of 60 dimers of VP3 (100 kDa). The VP3 layer encloses a viral genome of 10 dsRNA segments of discrete sizes (S1-S10), together with the transcription complex of three proteins, VP1, VP4 and VP6 termed the subcore. The genome is ˜19 kb in size, separated into 10 individual segments, S1 to S10 (0.8 to 3.9 kb), which encode 7 structural (VP1-VP7) and 4 non-structural (NS1-4) viral proteins, each of which is involved in various stages of the virus replication cycle. The ten segments of BTV vary both in sequence and size (from 0.8 kb to 3.95 kb) but are clustered in three distinct size classes (large, S1-S3; medium, S4-S6 and small, S7-S10).

BTV enters mammalian cells via an endocytic pathway where the particle is uncoated (removal of VP2 and VP5) to release core particles into the cytosol. The genomic dsRNA segments are never released from the core; rather the intact core produces 10 capped mRNAs (ssRNAs), one from each genomic segment, which are repeatedly synthesised by core-associated enzymes and extruded into the cytoplasm via pores in the 12 vertices of the icosahedral structure. These newly synthesised ssRNAs serve as messenger RNAs (mRNAs) that express all viral proteins but they also serve as templates following packaging into newly formed viral cores where the synthesis of genomic dsRNAs subsequently takes place. In BTV-infected cells, ssRNA packaging and the assembly of cores occurs within virus-induced inclusion bodies (VIBs), a matrix structure that is enriched with NS2 protein. NS2 has been shown to have sequence-specific affinity for BTV ssRNA segments and it also recruits BTV ssRNAs into the viral assembly location in BTV-infected cells. However, we have recently shown that NS2 is not necessary for in vitro ssRNA packaging.

Some segmented RNA genome viruses appear to have a non-selective packaging mechanism, for example, the two segments of infectious bursal disease virus genome are randomly packaged into virions and produce a large proportion of non-infectious particles with incomplete genome. However, we suggest it is mathematically impossible for the members of Reoviridae, containing 9 to 12 genome segments, to adopt this mechanism as the percentage of infectious particles containing complete genome would be too small. Moreover, the particles to infectivity ratio for these viruses is high, also in our view suggesting that there must be a selective packaging mechanism allowing the multiple genomic segments to correctly and effectively package into newly formed capsids.

Here, we show for the first time that there is a packaging order for how ssRNA viral segments can be packaged into their protective coats, or capsids, during infection and that this can be exploited for the purpose of controlling viral replication and so infection.

### STATEMENTS OF THE INVENTION

According to a first aspect of the invention there is provided a pharmaceutical composition effective against a member of the Reoviridae family and comprising at least one oligonucleotide complementary to, and so able to bind with, an untranslated region (UTR) of nucleic acid located, either 5′ or 3′, adjacent the coding region of at least one of the viral genome segments that constitutes the viral genome together with at least one pharamaceutically acceptable carrier.

In a preferred embodiment of the invention said viral genome segment constitutes the smallest segment within the viral genome or the smaller of the total segments within the viral genome having regard to size and being typically, but not exclusively categorised as small (S) within the Reoviridae.

Additionally, or alternatively, said oligonucleotide is complementary to, and so binds with, the longest UTR of nucleic acid located, either 5′ or 3′, adjacent the coding region of at least one of the viral small (S) genome segments, typically but not exclusively the smallest viral (S) genome segment that constitutes the viral genome.

Most ideally, said oligonucleotide is complementary to, and so binds with, the UTR of nucleic acid located, either 5′ or 3′, adjacent the coding region of said viral genome segment selected from the group comprising S6, S7, S8, S9, S10, S11 and S12.

More preferably, said untranslated region of nucleic acid is located 3′ of said coding region. Alternatively, said untranslated region of nucleic acid is located 5′ of said coding region.

We demonstrate herein that among the Reoviridae family, for example in the Blue Tongue Virus (BTV), of all the genomic segments the smallest segment (such as S10 in BTV) with the longest UTRs (5′ UTR and 3′ UTR together=155 bases) initiates RNA packaging. We show herein that S10 plays a crucial role in initiating genome packaging within the viral capsid by representing the fundamental packaging member that recruits the other smaller RNA segments to form a complex or complexes that can then interact with the larger genome segments.

In other words, we show that ordered RNA-RNA interactions are required for packaging the RNA segments in segmented dsRNA viruses.

As used herein, the term “oligonucleotide” describes an oligonucleotide that is an oligoribonucleotide, oligodeoxyribonucleotide, modified oligoribonucleotide, or modified oligodeoxyribonucleotide which hybridizes under physiological conditions to viral RNA comprising an UTR located adjacent a particular viral gene segment and thereby, inhibits the function of the bound RNA and so prevents viral packaging in the viral capsid.

The term “modified” also encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-0-alkyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include alkylated purines and/or pyrimidines;

acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5 carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; □-D-mannosylqueosine; 5-methoxycarbonylmethyluracil; 5-methoxyuracil; 2 methylthio-N6-isopentenyladenine; uracil-5-oxyacetic acid methyl ester; psuedouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Modified double stranded nucleic acids also can include base analogs such as C-5 propyne modified bases (see Wagner et al., Nature Biotechnology 14:840-844, 1996). The use of modified nucleotides confers, amongst other properties, resistance to nuclease digestion and improved stability.

Those skilled in the art will recognize that the exact length of the oligonucleotide and its degree of complementarity with its UTR target will depend upon the specific UTR target selected, including the sequence of the target and the particular bases which comprise that sequence.

It is preferred that the oligonucleotide be constructed and arranged so as to bind selectively with the UTR target under physiological conditions, i.e., to hybridize substantially more to the target sequence than to any other sequence in the target cell under physiological conditions.

In order to be sufficiently selective and potent for inhibition, such oligonucleotides should comprise at least 7 (Wagner et al., Nature Biotechnology 14:840-844, 1996) and more preferably, at least 12, 13, 14, or 15 consecutive bases the majority of which are complementary to the target. Most preferably, the oligonucleotides comprise a complementary sequence of 20-30 bases. Most preferably still, the oligonucleotides comprise a complementary sequence of bases to the entire 5′ or 3′ UTR.

Most preferably the oligonucleotide of the invention is, in ascending order of preference, at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical with the target UTR with which it is complementary.

The term oligonucleotide is to be construed as a material manufactured either in vitro using conventional oligonucleotide synthesising methods which are well known in the art or oligonucleotides synthesised recombinantly using expression vector constructs.

Ideally said selected viral genome segment(s) is/are the smallest or smaller in the viral genome. Ideally, any one or more of S7-10 in BTV; any one or more of S7-10 in AHSV; any one or more of S6-11 in Rotavirus; and any one or more of S6-12 in Colorado Tick Virus.

In yet a more preferred embodiment of the invention said oligonucleotide is complementary to, and so able to bind with, at least a part of a UTR selected from Table 1.

In a further preferred embodiment of the invention said oligonucleotide is complementary to the whole ora part of SEQ ID Nos: 9, 10 11, 12, 13, 14, 15, 16, 17, 18 and 19.

In a preferred embodiment of the invention, said Reoviridae virus is selected from the group comprising: Cardoreovirus, Mimoreovirus, Orbivirus, Phytoreovirus, Rotavirus, Seadornavirus, Aquareovirus, Coltivirus, Dinovernavirus, Idnoreovirus and Mycoreovirus.

More preferably still, said Reoviridae virus is selected from the group comprising: Rotavirus such as the Colorado tick virus (neurologic disease), Reovirus such as Aquareviruses (infects fish and mollusks), fusogenic orthoreviruses (causes pulmonary disease in human), orbiviruses (African horse sickness, kills horses), Bluetongue (infects sheep and cattle), Seadornavirus (infects human), Avian reovirus and Rice dwarf virus (Phytoreovirus).

In yet a further preferred embodiment of the said oligonucleotide is selected form the group comprising:

BTV Segment 9
(SEQ ID No: 22)
UGACAUAUGCGAUUUUUUAAC
(SEQ ID No: 23)
GUAAGUGUAAAAUCGCCCUACGUCAAGAAGGUA
(SEQ ID No: 24)
UUAGAGGUGAUCGAUCAAAUGCAGGAACUCCGUUUUCACA
(SEQ ID No: 25)
CUUCUGUUAGAACUACCCAUCUUCCUCCAUUCGCUCC
BTVSegment 10
(SEQ ID No: 26)
AUCAGCCCGGAUAGCAUGGCAGCGACACUUUUUAAC
(SEQ ID No: 27)
GUAAGUGUGUAGCGCCGCAUACCCTCCCCCGUUAGACAGCA
(SEQ ID No: 28)
CCUCGGGGCGCCACUCUACCUACUGAUCUUAGGUUAAUG
(SEQ ID No: 29)
UUAGGUUAAUGGUAAUUCGAAACCAUCUAGCGGGA
(SEQ ID No: 30)
AAUUUGCUGGUUCAAGCUUCUCUCGCUUUUUGCGC
(SEQ ID No: 31)
GTAGGAGTCTGCATCGTGAGATCAACCACTCTAC
(SEQ ID No: 32)
UGCUAUUACCAUGCUACAGAUGUAAGUGAU

The pharmaceutical composition of the invention may comprises a plurality of said oligonucleotides each one of which is complementary to, and so able to bind with, an untranslated region of nucleic acid located, either 5′ or 3′, but ideally 3′, adjacent the coding region of at least one of the viral genome segments that constitutes the viral genome. In preferred embodiments of the invention said oligonucleotides are designed to target both the 5′ and 3′ UTR of at least one selected viral genome segment and in yet a further preferred embodiment said oligonucleotides are designed to target a UTR of a plurality of selected viral genome segments, more preferably yet, said oligonucleotides are designed to target both the 5′ and 3′ UTR of said plurality of selected viral genome segments.

The present invention, thus, includes pharmaceutical compositions containing natural and/or modified oligonucleotide molecules that are complementary to and hybridize with, under physiological conditions, an untranslated region of nucleic acid located, either 5′ or 3′, adjacent the coding region of at least one of viral genome segments that constitutes the viral genome, together with at least one pharmaceutically acceptable carrier (e.g. polymers, liposomes/cationic lipids).

Preferably, the pharmaceutical composition may include the oligonucleotide(s) in combination with any standard physiologically and/or pharmaceutically acceptable carriers which are known in the art (e.g. liposomes). The compositions should be sterile and contain a therapeutically effective amount of the oligonucleotides in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism.

Oligonucleotides may be administered as part of a pharmaceutical composition.

Formulations for administration include those suitable for oral, rectal, nasal, bronchial (inhaled), topical (including eye drops, buccal and sublingual), vaginal or parenteral (including subcutaneous, intramuscular, intraperitoneal, intravenous and intradermal) administration and may be prepared by any methods well known in the art of pharmacy.

The route of administration will depend upon the condition to be treated but preferred compositions are formulated for intravenous, parenteral, oral, nasal, bronchial or topical administration.

The composition may be prepared by bringing into association the oligonucleotide of the invention and the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a oligonucleotide of the invention in conjunction or association with a pharmaceutically or veterinarily acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, formulations may be made into a cream, ointment, jelly, solution or suspension etc. Cream or ointment formulations that may be used for the drug are conventional formulations well known in the art, for example, as described in standard text books of pharmaceutics such as the British Pharmacopoeia.

Oligonucleotides of the invention may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

The precise amount of a oligonucleotide of the present invention which is therapeutically effective, and the route by which such compound is best administered, is readily determined by one of ordinary skill in the art by comparing the tissue level of the agent to the concentration required to have a therapeutic effect.

According to a further aspect of the invention there is provided a combined pharmaceutical composition comprising a pharmaceutical composition according to the invention and one or more different additional anti-viral agents.

Those skilled in the art will appreciate that the additional anti-viral agents are conventionally known.

According to a second aspect of the invention there is therefore provided a method for treating a viral infection comprising administering to an individual to be treated an effective amount of a pharmaceutical effective against a member of the Reoviridae family and comprising an oligonucleotide complementary to, and so able to bind with, an untranslated region of nucleic acid located, either 5′ or 3′, adjacent the coding region of at least one of the viral genome segments that constitutes the viral genome.

According to a further aspect of the invention there is therefore provided a method for treating a viral infection comprising administering to an individual to be treated a pharmaceutical composition according to the invention.

According to a yet further aspect of the invention there is provided the pharmaceutical composition of the invention to treat a viral infection.

According to a yet further aspect of the invention there is provided use of the pharmaceutical composition of the invention in the manufacture of a medicament to treat a viral infection.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

An embodiment of the present invention will now be described by way of example only with reference to the following wherein:

### FIGURE LEGENDS

FIG. 1. Exclusion of specific BTV RNA segment influences genome packaging. An incomplete set of ³²P-labelled BTV ssRNAs that excludes S2 or S5 or S10 (indicated as −S2, −S5, −S10) but includes all the 9 respective segments were used in the in vitro CFA assay; the reaction mixture was purified on a sucrose gradient. A complete set of 10 ssRNAs was also included (all 10) as a control. (A) RNA distribution within the sucrose gradient fractions 2 to 9 was analysed on an agarose gel. The fraction containing assembled cores (fraction 6) is indicated with an asterix (*). (B) Packaged RNA profile after RNAse digestion and purification of fraction 6 analysed by 1% denaturing agarose gel. Segments S1 to S10 are indicated on the left. (C) Quantification of the effect of segment exclusion. 10 BTV ssRNAs (WT) or ssRNAs excluding one ssRNA at a time (−S1, −S2, etc.) were used in the CFA assay. Packaged ssRNA in relevant fraction containing cores was purified on a sucrose gradient. BTV segment S6 was quantified by qRT-PCR to represent the packaging efficiency. Quantities of S6 in samples of −S1, −S2 etc. were compared with WT control in the same experiment and packaging efficiency was calculated. Standard deviations from three independent experiments are indicated (error bars).

FIG. 2. Exchanging UTRs of segment S10 influences packaging in vitro. (A) Schematic representation of chimeric segments S10. The UTRs of BTV-1 S10 (white boxes), flanking the open reading frame (grey boxes) were replaced with UTRs (pattern boxes) of BTV-1 S3 (S3/S10) or S5 (S5/S10) or S8 (S8/S10) or BTV-10 S10 (B10/B1) or AHSV-4 S10 (A4/B1) using overlap PCR and T7 transcription. (B) BTV-1 S10 (WT) or chimeric S10 (S3/S10, or S5/S10, or S8/S10, or B10/B1, or A4/B1) were included with segments 51 to S9 in CFA assay. Packaged RNAs were purified and the encapsidation of S10 in each assay was measured by qRT-PCR using specific primers for the BTV-1 S10 coding region. The packaging efficiency and standard deviations (error bars) for each condition was calculated and normalised considering VVT conditions as 100%.

FIG. 3. Effect of exchanging UTRs of S10 in packaging using an in vivo system. (A) A cartoon shows the process of in vivo single replication packaging assay. A modified sequence that can be specifically detected and quantified by PCR (marked with *) was introduced in the chimeric S10 ssRNAs used for transfection (in dark grey). 12 to 16 hours after transfection and infection, newly formed cores were purified and the amount of modified RNA packaged within the cores was measured. (B) Quantification of modified S10 (VVT, S3/S10, S5/S10, S8/S10, B10/B1 and A4/B1) packaged in the new viral cores was correlated with the total quantity of new cores in the sample to obtain the packaging efficiency. The data was standardised to the wild-type data considered to be 100% and the ratios were calculated. Standard deviations are indicated as error bars.

FIG. 4. Effects of chimeric S10 on virus recovery using reverse genetics system. A complete set of 9 BTV-1 segments (S1 to S9) and one of the chimeric S10 (S3/S10 to A4/B1) or the VVT S10 were used in reverse genetics system. (A) Recovered viruses were amplified once and analysed by plaque assay on monolayers of BSR cells. (B) Genomic dsRNA of reassortant virus containing BTV-10/BTV-1 chimeric S10 (B10/B1) was purified and the sequence was confirmed by RT-PCR and sequencing. Nucleotides specific to BTV-10 in the electropherogram and in the actual sequence are indicated with an asterix. The sequencing data shows the reverse complement strand. (C) Deletion in UTRs suppresses packaging. BTV-1 S10 (WT), 5′ UTR and both UTRs truncated S10 (Δ5UTR and ΔUTRs), and a series of 3′ end truncated S10 (Δ12, Δ35, Δ60) were tested using CFA assay as described in FIG. 2B.

FIG. 5. A schematic for RNA-RNA interaction assay based on BTV S10 coated beads.

FIG. 6. BTV S10 interacts with smaller segments. (A) Beads coated with a S10 specific primer were incubated sequentially with BTV-1 S10 and with 1 pmol of segments S1 to S9 individually. Interaction with RRV S9 was included as a negative control. After extensive washing, the attached RNA was released by heating. The amount of interacting RNA was determined by qRT-PCR using primers specific to each segment. The copy number was correlated by minus non-specific binding detected in beads-only control. S10 and the standard deviations from three individual experiments are indicated. (B) Beads coated with (+) or without (−) BTV-1 S10 were similarly prepared and sequentially incubated with ³²P-labelled S1, S3, S6 or S8. After three washes, RNAs were heat-released and analysed on a denaturing agarose gel and Phospho-imager exposure. The black arrows indicate positive interaction. (C) The interaction between S8 and truncated S10 was measured with a similar method: beads were coated with BTV-1 S10 (S10), S10 lacking 5′ and 3′ UTRs (ΔUTRs), 3′ UTR (Δ3′UTR) or 5′ UTR (Δ5′UTR) and incubated with equal amounts of BTV-1 S8. Interacting S8 was analysed and quantified similarly. Interaction rates and standard deviation (error bars) were calculated.

FIG. 7. Smaller segments link larger segments to S10 in a specific order. S10 coated beads were prepared as described in FIG. 6. (A) 1 pmol BTV-1 S1, S5 or RRV S9 were incubated with S10 beads only (S10) or together with a mixture of S6, S7, S8 and S9 (S6-S10). Interacting ssRNAs were analysed by qRT-PCR and correlated to the control lacking S10. Standard deviations from three individual experiments are indicated (error bars). (B) Enhancement of the interaction between S5 and S10. BTV-1 S5 was incubated with S10 beads alone (S10) or with S6, S7, S8 or S9 separately (S10+S6 etc.) or with a mixture of S7 to S9 (S10+S7-S9) or S6 to S9 (S10+S6-S9). (C) The interaction between S1 and S10 was enhanced when adding a different mixture of segments. BTV-1 S1 was incubated with S10 beads alone (S10) or with a mixture containing S6 to S9 (S6-S10), or S4 to S9 (S4-S10) or S2 to S9 (S2-S10).

FIG. 8. Specific interaction order among segments with different sizes. (A) Smaller segments link larger segment S1 to S8. S8 coated beads were prepared similarly as S10 coated beads as described. BTV-1 S1 was incubated with S8 beads alone (S8) or with mixtures of S8 to S10 (S8-S10), or S4 to S10 (S4-S10), or S2 to S10 (S2-S10). The pulled-down S1 was quantified with qRT-PCR as described. (B) Larger segments also link smaller segment S10 to S3. S3 coated beads were similarly prepared. BTV-1 S10 was incubated with S3 beads alone (S10) or with different mixtures (S1-S3, or S1-56, or S1-59). The increased interaction was shown in bar and standard deviation (error bars) were calculated.

FIG. 9. Complete set of BTV RNA segments is required for RNA packaging. Full set of BTV-1 10 ssRNAs (WT), S4 to S10 (S4-S10), S6 to S10 (S6-S10), or S10 only were used in CFA system to determine the packaging efficiency as described in FIGS. 1 and 2. Three segments (S4, S7, and S10; shown in different patterned bars as indicated) were detected with qRT-PCR when applicable. The packaging efficiencies were shown in percentage and standard deviation (error bars) were calculated.

FIG. 10. In vivo effect on virus replication of antisense ORNs complementary to Rotavirus S10 and S11. A. Representative examples of plaque assay stained with crystal violet (E). Histogram of virus yield in the presence of different ORNs. S10, S11, AUG or Scr ORNs were transfected to MA104 monolayer cells for 3 hours followed by infection with Rhesus Rotavirus at 0.1 MOI with DMEM without FCS+0.8 ug/ml trypsin. At 48 hpi virus yield was determined by plaque forming units (PFU). Values (%) represent the mean and standard deviation of the mean (n=3) generated relative to the control (without ORNs) set at 100%.

FIG. 11. In vivo effect of antisense ORNs complementary to S1, S9 and S10 on virus replication. Schematic representation of S1(A), S9(B) and S10(C) indicating the 5′ and 3′UTRs and the protein coding region (ORF) with the initiation codon (AUG). Positions targeted by the antisense ORNs and 3′UTR length are indicated in each case (D). Representative examples of plaque assay stained with crystal violet (E). Histogram of virus yield in the presence of different ORNs. S1, S9 and S10 or Scr ORNs were transfected to BSR monolayer cells for 3 hours followed by infection with BTV-1 at 0.1 MOI. At 24 hpi virus yield was determined by plaque forming units (PFU) as described in Material and Methods. Values (%) represent the mean and standard deviation of the mean (n=3) generated relative to the control (without ORNs) set at 100%.

FIG. 12. Translation efficiency of BTV mRNA in the presence of ORNs. Synthesized VP1 (A), VP6 (B) and NS3/NS3A (C) viral proteins in the absence or presence of antisense ORNs in different concentration (μM). Positions of molecular mass standards are indicated in kDa. (D) Histogram of virus yield and cell-free translation in the presence of different ORNs are indicated. Translation efficiency values were calculated by densitometry as the ratio of the translated product relative to the ‘No ORN’ control, set as 100%. Virus yield was expressed as the reduction of the number of plaques (PFU). Values (%) represent the mean and standard deviation of the mean (n=3).

FIG. 13. RNA-RNA interactions between small BTV segments. (A) Independently transcribed and purified RNA segments (lanes 1 to 4) were heated for two minutes prior incubation in pairs (lanes 5 to 10) as indicated. Themobility shift was analysed by native agarose gel. Interactions are indicated on the right. (B) Simultaneous co-transcription of multiple segments (lanes 5 to 15) as indicated. The positions of the retarded complex and free RNA are indicated on the right (upper panel). Purified and co-transcription reactions, RNA-RNA complexes were quantified by densitometry and expressed as percentage of the ratio of bound and unbound RNA (lower panels of A and B). Values (%) are from the mean and the standard deviation of >3 independent assays (n=3-5). Individually transcribed segments are shown (lanes 1 to 4,upper panels A and B) as controls. (C) For specificity, multiple co-transcription in the presence of different amounts of yeast tRNA are shown. The position of the bound (retarded RNA complexes) and unbound free RNAs are indicated.

FIG. 14. Mobility shifts of RNA complexes in the presence of ORNs. (A) Combinations of three or four BTV RNA segments were co-transcribed in the presence (+) or absence (−) of S10.2 (lanes 5 to 14) or S10.4 (lanes 15 and 16) ORNs. The bound RNA complexes and unbound RNA are indicated (upperpanel) and quantification shown in histogram (lowerpanel). (B) The effect of Scr, S10.1 and S10.5 ORNs on RNA complex formation was similarly analysed and presented. (C) Histogram of the percentage of the RNA complex in each lane of A and B was determined against the total mass of input RNA. The RNA complexes in presence of ORNs were normalized relative to the control complexes without ORNs. Values (%) represent the mean and standard deviation of >3 independent assays (n=3-5). (D) RNAs from co-transcription reactions in the presence or absence of S10.2 analyzed on a 1% denaturing agarose gel.

FIG. 15. Effect of deletions in S10 on RNA-RNA interactions. (A) Schematic representation of S10 depicting deleted sequences (ΔS10.2 and ΔS10.5) as indicated. (B) Mobility shift assay of co-transcription complexes in the presence of ΔS10.2 and ΔS10.5 mutants (lanes 7 to 18). Position of retarded complexes and free RNA are indicated and quantification of bound to unbound RNA are shown (lanes 7-18, lower panel). The RNA complexes in each lane with S10 WT or each mutant were determined against the total mass of input RNAs as (%). The RNA complexes with S10 mutants were normalized relative to the complexes formed with the VVT S10. Values (%) represent the mean and the standard deviation of >3 independent assays (n=3-5). (C) Simultaneous or individual RNA transcriptions in the presence or absence of ΔS10.2 or ΔS10.5 analyzed in a 1% denaturing agarose gel.

FIG. 16. Effect of ORN on RNA packaging in cell-free assembly assay. 35S-labelled in vitro assembled BTV complexes were fractionated in a continuous sucrose gradient (Upperpanel): Fractions #5, #6 and #7 from cell-free assembly (CFA) reactions in the absence (+control, lanes 1 to 3) or presence of 20 pmol S10.2 ORN (lanes 4 to 6) along side with fraction #6 in the presence of S10.4 ORN (lane 7) S10.5 (lane 8) and Scr ORN (lane 9) were analysed on 1% denaturing agarose gel. Packaged RNAs were determined by densitometry. Lower panel represents the mean values (%) of total packaged ssRNAs in the presence of ORNs calculated relative to the control reaction (without ORNs) set at 100% (n=3).

FIG. 17. Designed mutations used in reverse genetics system. [A] Schematic map of S10 showing the position of substitution mutations on regions encompassed by S10.2 and S10.5 ORNs in the 3′UTR. [B] Sequences (5′-3′) of S10 substitution mutants, position of each in relation to different ORNs and their effects on virus recovery by RG system. RG results and percentage of cells exhibiting CPE are indicated. Copy number of genomic segment S6 was used to quantify the amount of released viral particles.

Table 1. Nucleic acid sequences of the 5′ and 3′ Untranslated Regions either side of the coding sequence for the genome segment indicated in Bluetongue virus BTV-1 and BTV-10 (rows 1-2); AHSV-4 row 3; Rotavirus C (row 4); Colorado tick virus (rows 5-6).

Table 2. The 2′O-methyl modified antisense oligoribonucleotides (ORNs) used for in vivo and in vitro studies. The ORN name, sequence (5′-3′), length, target BTV RNA segments regions are listed.

Table 3. Summary of RNA-RNA interactions studies. Left panel: Interactions between two RNA segments of purified or co-transcribed RNAs. Right panel: Interactions among three or four segments by co-transcription. Values (%) are from mean and standard deviation of >3 independent experiments (n=3-5).

Table 4, Interactions of multiple BTV segments in the presence or absence of ORNs (left panel) and S7/S8/S9 with S10 VVT or S10 with deletion mutants (right panel). Values (%) are the mean and standard deviation of >3 independent experiments made relative to control (no ORN or S10 WT) set at 100% (n=3-5).

Table 5. Summary of the inhibitory effects of ORNs. Plus (+) sign indicates inhibitory effects and negative (−) non-inhibitory effects deduced from different assays performed.

### Materials MATERIALS AND METHODS

Plasmids and DNA templates. To generate T7 transcripts, template plasmids containing a T7 promoter and a specific restriction enzyme site flanking cDNA of exact copies of each BTV-1 genome segment (South African reference strain, Genbank accession numbers FJ969719-FJ969728), BTV-10 S10 (U.S. isolate, NC006015), AHSV-4 S10 (FJ183368), and Rhesus Rotavirus (RRV) S9 (EU636932.1) derived from viral dsRNA using the method of full-length amplification of cDNA (FLAC) were used. Chimeric S10 constructs were generated using 5′ primers encoding T7 promoter and 3′ primers (available upon request). A sequencing marker, replacing the sequence of 384-399 nt from 5′-GTTGAAAAGTGACCTA-3′ (SEQ ID No: 1) to 5′-ACTAAAGAGCGATTTG-3′ (SEQ ID No: 2) was also introduced in each chimeric S10 construct.

For the generation of S10 RNA deletion mutants, two S10 deletion constructs corresponding to the target sequences of S10.2 (39 nts) and S10.5 (34 nts) ORNs were generated by polymerase chain reaction (PCR) through site-directed mutagenesis (37). Amplicons were then treated with Dpnl to digest the parental plasmid prior to transformation into competent cells. For the generation of four S10 RNA substitution mutants S10.2₇₁₃₋₇₁₈, S10.2₇₂₅₋₇₃₀, S10₇₂₈₋₇₃₂ and S10.5₇₄₃₋₇₄₈ site directed mutagenesis was performed by overlapping PCR using S10 specific primers. Deletion and interchanging 3′UTRs of S8 and S10 were also generated by overlapping PCR followed by Dpn 1 treatment. Capped BTV RNA transcripts for in vitro translation assay were generated using mMESSAGE mMACHINE® Kit (Ambion) as described previously. For generation of uncapped ssRNA for cell-free assembly, linearized DNA were incubated at 37° C. for 2 h with 40 U of T7 RNA polymerase (Thermo Scientific), 50 mM DTT, 0.5 mM each rNTP and 10 U RNase inhibitor (Thermo Scientific).

### Cells and Virus

Bluetongue virus serotype 1 (BTV-1) South African reference strain was plaque purified and amplified in BSR cells, a BHK 21 clone derivative of baby hamster kidney cells (American Type Culture Collection) grown in Dulbecco modified Eagle medium containing 5% fetal calf serum (FCS) penicillin, streptomycin and amphotericin B at 35° C. with 5% CO₂. Virus stocks were maintained by infecting BSR cells at multiplicity of infection (MOI) of 0.1 and harvested at 48-72 hpi.

Generation of T7 transcripts. Capped and uncapped ssRNAs were generated as previously described (17) using mMessage RNA (Ambion) and T7 High (Thermos) respectively.

Cell-free in vitro assembly assay. Packaging of viral ssRNAs was investigated using a recently established cell-free assembly (CFA) assay (8). Packaging efficiency was estimated using either ³²P-labelled ssRNAs or non-radioactive qRT-PCR. For ³²P-labelled ssRNAs, T7 transcripts were 3′ end-labelled with 10 μCi 5′-³²P-cytidine (Perkin-Elmer) using T4 RNA ligase (Fermentas). The CFA assay was carried out as described previously (8). Briefly, VP1, VP4, VP6, VP3 and VP7 were sequentially in vitro translated from capped ssRNA of coding regions, followed by incubation with full-length 10 BTV uncapped ssRNAs to allow viral core assembly. The whole mixture was loaded onto a continuous sucrose gradient and fractions were collected after ultracentrifugation. In the relevant fraction (fraction 6), unpackaged RNAs were eliminated by RNase One (Promega) digestion. Packaged RNA was extracted and analysed by denaturing agarose gel electrophoresis. To detect radiolabelled RNA, the gel was dried and exposed to a Storage Phosphor screen and analysed with Phosphor-imager and ImageQuantTL software (GE Healthcare).

The cell-free system for BTV was carried out as described (10) with some modifications. Briefly, VP1, VP4 and VP6 were synthesized from RRL system followed by incubation with the complete set of 10 full-length (300ng each) uncapped ssRNAs with or without 20 pmol S10.1, S10.2, S10.4, S10.5, S10 AUG and Scr ORNs. In vitro synthesized VP3 and VP7 were then added to the mixture and further incubated to allow viral core assembly. After eliminating unpackaged RNA by RNase One (Promega) digestion, the assembled particles in the reaction mixture were isolated by a 15% to 65% continuous sucrose gradient followed by fractionation as described previously (10). For positive control, S10.2 and S10.5 ORN gradients, packaged RNAs were extracted from fractions 5, 6 and 7 and analysed by denaturing 1% agarose gel electrophoresis to identify the packaged 10 ssRNAs (10). Only fraction 6 was collected for samples with S10.1, S10.4, S10.5, S10 AUG and Scr (packaged ssRNAs are previously shown to be present at this fraction) (10). For analysis of in vitro incorporated proteins, the in vitro synthesized viral proteins were radio labelled with 35S-methionine, analysed in 9% SDS-PAGE and detected by autoradiography.

Quantitative RT-PCR. For detection of non-radioactive ssRNA, BTV-1 S6 or chimeric S10 were analysed by qRT-PCR using either primers reported by Toussaint et al (18) or BTV-1 S10 335F: 5′-GTTGAAAAGTGACCTAGGAGGC -3′ (SEQ ID No: 3) and BTV-1 S10 492R: 5′-TTCACCACACCTAACATTGGG -3′ (SEQ ID No: 4), respectively. BTV RNAs from the packaging assay were reversely transcribed (RT) into cDNA using ReverseAid Premium Reverse Transcriptase (Thermo) and quantified with suitable primers using 7500 Fast Real-Time PCR system and SYBR select Master Mix (Applied Biosystems). Three independent experiments were undertaken and qPCR was performed in duplicate. Standard deviations from the three experiments were calculated.

In vivo Packaging Assay. 10⁶ BSR cells were transfected as previously described (19) with 2 μg of uncapped T7 transcripts of wild-type or chimeric S10. The cells were subsequently infected with BTV-1 at a multiplicity of infection (MOI) of 3. After 12 h, allowing for one replication cycle to be completed, cells were lysed and aliquots were stored for transfection control. Viral cores were then purified from the major portions of lysates as previously described. Unpackaged RNAs were digested with RNase at a final concentration of 1 μg/μl. Viral genomic RNA was then extracted, precipitated and subjected to qRT-PCR with a primer specific for the marker sequence (5′-ACTAAAGAGCGATTTG-3′) (SEQ ID No; 2) located in non-UTR or BTV-1 S10: BTV-1 S10 marker R: 5′-CCCAAATCGCTCTTTAG-3′ (SEQ ID No: 5). Copy number of marked S10 was correlated to the total BTV-1 S6 representing the number of total viral cores. Transfection discrepancy was further correlated with the copy number of marked S10 detected in the stored cell lysate aliquots.

Reverse Genetics (RG) System. To generate the virus with chimeric S10, BSR cells were firstly transfected with pCAG plasmid encoding primary replication complex (VP1, VP4 and VP6), VP3 and NS2 as described previously (17), followed by a second transfection with capped chimeric S10 ssRNA together with the remaining 9 BTV-1 ssRNAs. Replication of recovered viruses was visualised by plaque assay. To confirm the recovery of mutant virus, genomic dsRNAs were purified from the infected cells, reverse transcribed and the integrity of chimeric S10 was confirmed by nucleotide sequencing (Source Bioscience).

To generate the virus with S10 mutants (S10.2713-718, S10.2725-730, S10728-732 and S10.5743-748, and chimeric S10 and S8) BSR cells were transfected with mutated S10 ssRNA together with the remaining 9 BTV-1 ssRNAs as described previously (12, 40). For combined chimeric S10 and S8, BSR cells were transfected with mutated S10 ssRNA together with the remaining 8 BTV-1 ssRNAs. Replication of recovered viruses was visualised by crystal violet staining. Virus recovery was quantified by qRT-PCR using specific BTV genomic primers as previously described (9). To confirm the recovery of mutant virus, genomic dsRNAs were purified from the infected cells, reverse transcribed and the mutated sequences of S10 was confirmed by nucleotide sequencing (Source Bioscience).

RNA interaction assay. ssRNA of BTV-1 S10 was attached to beads leaving its 5′ and 3′ ends free by the following methods: Streptavidin agarose beads (Novagen) were coated with a biotin-labelled primer which annealed to nt 401-700 in the coding region of S10 (5′- biotin-TTTTTTTTTTTGTATTAT AGCTCTTTTCTTCTTTAAGCCTC -3′) (SEQ ID No: 6). The beads were incubated with poly-A RNA to decrease non-specific binding. BTV-1 S10 was then incubated with the coated beads followed by the addition of other ³²P-labelled or non-labelled RNAs in an RNA folding buffer previously described (20). After 20 min incubation at 30° C., the beads were washed three times with excess folding buffer followed by 1 min heating at 90° C. to release the RNA. For the radiolabelled RNA assay, samples were analysed by a denaturing gel and phosphor screen exposure. For non-labelled RNA, samples were analysed by qRT-PCR using primers specific for the target RNA, as described above. The S8 coated beads and S3 coated beads were similarly prepared using the biotin-labelled primers: 5′-biotin-TTTTTTTTTTGC TTCATCATCATCCAGCGTGACTCTTCCCTTGGC -3′ (SEQ ID No: 7) for S8 beads and 5′-biotin-TTTTTTTTTTCAAC ATCTATTGTAGCCCATCCATTAT ATCCTGTTCCTG -3′ (SEQ ID No: 8) for S3 beads.

Design of Antisense Oligoribonucleotides with 2′O-Methyl Modifications Based on Prediction of ssRNA Structures

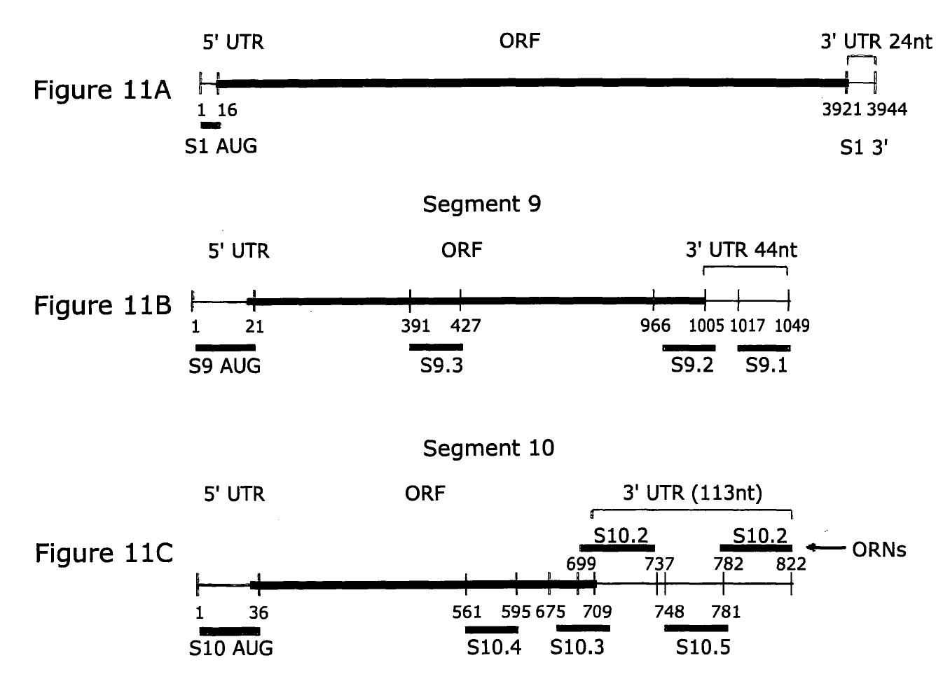

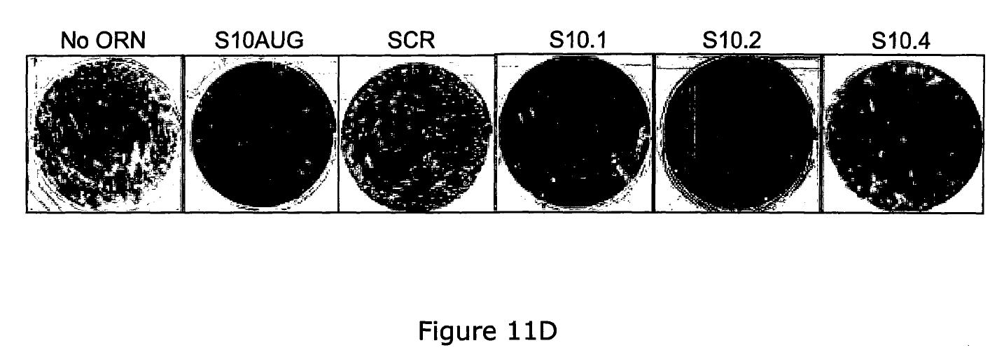

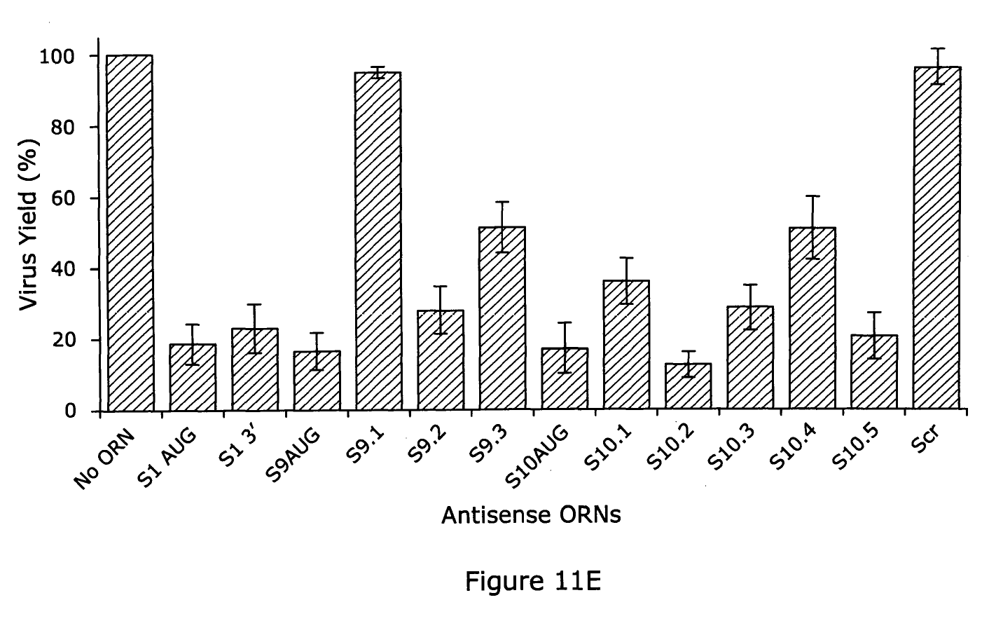

A series of thirteen antisense oligoribonucleotides (ORNs) were designed to hybridize either the 5′UTR including the AUG initiating codon, the internal coding region or the 3′ UTR of segments S1, S9 and S10 (Table 2). These ORNs were modified at the ribose with 2′O-methyl group (Integrated DNA Technologies) and named by their target position in each segment (FIG. 11). A scrambled (SCR) sequence of 30 nt, was included as specificity control. The scrambled sequence was verified by NCBI-BLAST software (http://blast.ncbi.nlm.nih.gov/) to prevent any possible match in the BTV genome or the host cellular RNAs. For the design of the ORN target sites the software Mfold (http://ma.thi.univie.ac.at/) and RNAfold (http://ma.tbi.univie.ac.at/cgi-bin/RNAfold.cgi) were used to predict the secondary structure and folding pattern of each RNA segments in the context of a full-length segment. OligoAnalyzer (http://eu.idtdna.com/calc/analyzer) was used to analyse each ORNs to avoid structures that might prevent its base-pairing to target RNA (perfect hairpin, self-dimerization and melting temperatures).

Optimization of Inhibitory Conditions of 2′OMe ORNs and Challenge with BTV-1

To determine the optimal inhibitory condition for each ORNs, a concentration range (0.5, 1.5 and 2.5 μM) of S10 AUG, S10 3′ UTR and SCR were transfected to BSR cells using Lipofectamine 2000 (Life Technologies). After 3 h incubation, the cells were infected with BTV-1 at MOI 0.1 for 1 h. The inoculum was removed by 3 washes with low pH medium (DMEM-HCl, pH 6) to inactivate free virus, twice with normal medium to restore pH and incubated with DMEM supplemented with 1% FCS and the appropriate ORNs for one virus replication cycle of 16-18 h. Cells were harvested and the virus titre was analysed by plaque assay. The virus yield was calculated as the mean of plaque forming units per ml (PFU/ml) of three independent transfection assays with each 2′OMe ORNs and expressed as the relative PFU/ml of BTV1 transfected without ORNs, consider as 100%. Cytotoxicity was determined by cell staining at the end of the treatment. The optimal concentration for the ORNs was 1.5 μM.

### In Vitro Translation in the Presence of 2′OMethyl ORN

Different concentration range (0.5, 2 and 4 μM) of ORNs S1 AUG, S1.3′, S9 AUG, S9.1, S9.2, S10.1, S10.2, S10.3, S10.5, S10 AUG or Scr were incubated with BTV transcripts (300 ng) for 20 min at 37° C. and added to a reaction mix containing 7.5 μl of nuclease-treated rabbit reticulocyte lysate (RRL, Promega), 1 mM amino acid mix minus methionine and 6 μCi ³⁵S-methionine. Translation reaction was incubated at 30° C. for 90 min and treated with 1 μl of 1 μg/μl RNase A for 10 min at room temperature. Labelled proteins were quantified by densitometry using PhosphorlmagerTM screen. The inhibition of BTV protein expression was calculated relative to the control lacking ORNs. The experiment was repeated at least three times.

### In Vitro Transcription for RNA-RNA Interaction Assays, RNA-RNA Interaction in the Presence of ORN and Electrophoretic Mobility Shift Assay

For RNA-RNA interactions of individual RNA segments, 1 μg of linearized plasmid was transcribed in a buffer containing 40 mM Tris-HCl pH 7.5, 10 mM MgCl2, 20 mM NaCl2, 3 mM spermidine, 50 mM DTT, 5 mM each rNTPs, 10 U RNase inhibitor and 40 U of T7 RNA polymerase (Thermo Scientific) for 3 hours at 37° C. followed by RNase free DNase 1 treatment. Transcribed RNAs were extracted by standard phenol-chloroform method and re-suspended in RNase free water. RNAs were individually heated at 80° C. for 1 min, ice chilled and mixed in pairs in folding buffer (50 mM Na cacodylate pH 7.5, 300 mM KCl and 10 mM MgCl₂) (38) and RNA-RNA complexes were allowed to form for 90 min at 30° C. and immediately analysed by electrophoresis in 1% agarose gel supplemented with 0.1 mM MgCl₂. Electrophoresis gel was run for 180 minutes min at 150 V in TBM buffer (45 mM Tris, pH 8.3, 43 mM boric acid, 0.1 mM MgCl₂) and stained with 0.01% (w/v) ethidium bromide. The integrity of transcribed RNA was checked by denaturing gel electrophoresis.

For co-transcription experiments, 150 ng linearized plasmid of each segments (S7-S10) were transcribed either in pairs or combinations of 3 to 4 plasmids (S7, S8, S9 and S10 or S10 mutants). RNA transcription was carried out in the same condition as individual RNA segments. Immediately after transcription and DNase 1 treatment, the reaction was analysed on a 1% agarose gel as described above. The percentage of the retarded RNA in each lane was determined against the total mass of input RNA (%) by densitometry (Gene Tools, Syngene). For RNA complex inhibition assay with ORNs, the simultaneous transcription of S7-S10 (combination of 3 or 4) was performed in the presence or absence of 20 pmol of S10.1, S10.2, S10.4, S10.5 and Scr ORNs and analysed as described above. Non-specific yeast tRNA (20 and 50 pmol) was incorporated in the co-transcription reaction as a control. Quantification of intermolecular RNA complex was performed as described above.

For RNA-ORN hybridization assay, 10 pmol of S9 AUG, S9.2, S10 AUG, S10.2, S10.3, S10.5 and Scr ORNs were 3′ end labelled with 10 μCi [³²P]pCp (Perkin Elmer) with T4 RNA ligase (Thermo Scientific) in T4 RNA ligase buffer and incubated at 4° C. overnight. Unincorporated ³²P was removed by exclusion chromatography (Illustra Microspin G-25 column, GE Healthcare). Prior to hybridization, unlabelled S10 RNA was denatured at 80° C. for 1 min, immediately chilled and then mixed with folding buffer (50 mM sodium cacodylate pH 7.5, 100 mM KCl and 10 mM MgCl₂). RNA-ORN hybridization was performed with 0.5 pmol of pre-folded S10 RNA annealed with ³²P labelled ORNs (1, 2 and 5 pmol of S9 AUG, S9.2, S10 AUG, S10.2, S10.3, S10.5 and Scr ORNs) in folding buffer in 10 μl final volume (39). The complex was allowed to form for 30 min at 30° C. followed by electrophoresis in 4% native acrylamide gel at 4° C. for 50 min. at 150V in TBM buffer, dried and exposed by autoradiography.

### Results

### The Smaller BTV RNA Segments Initiate Genome Pckaging.

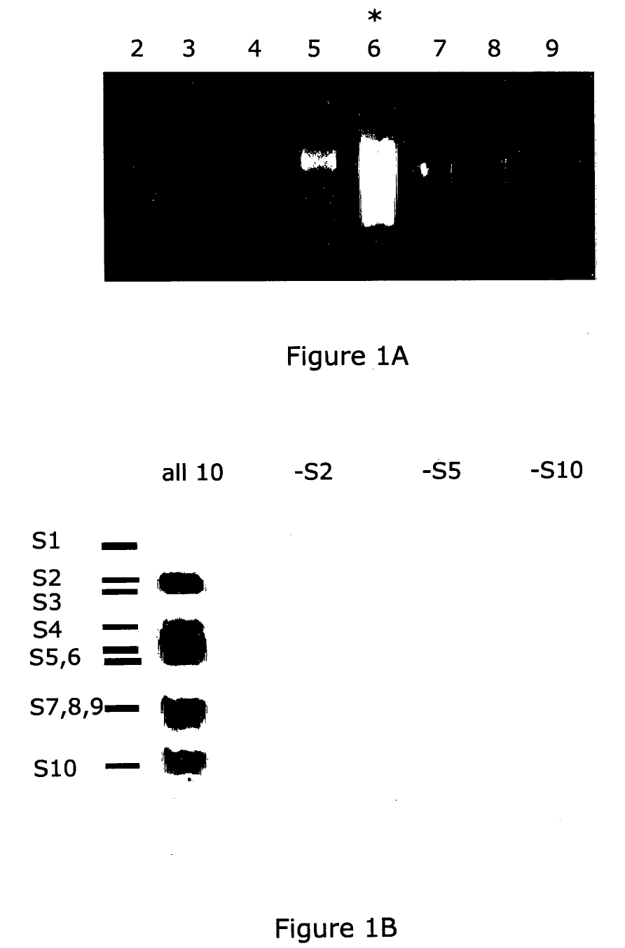

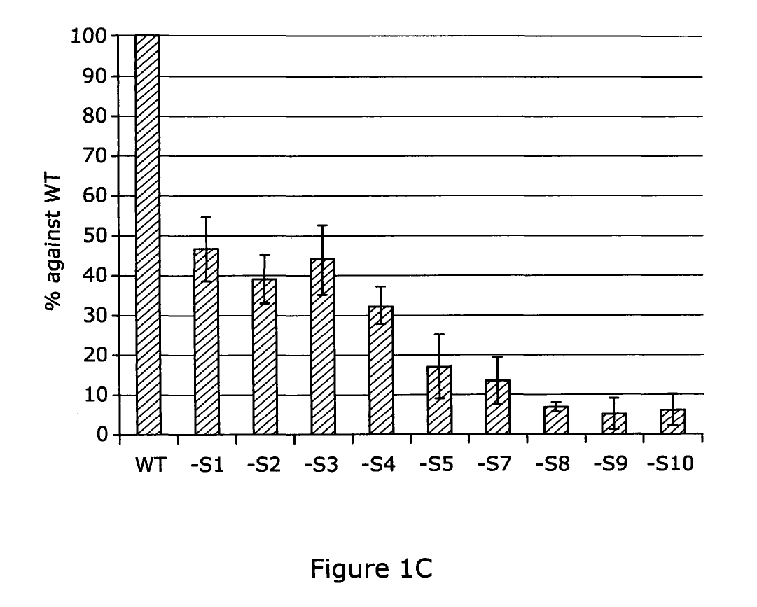

To investigate if there is a preferential packaging of BTV genome segments, we sequentially excluded one RNA segment from mixtures of the full 10 RNA segments and used the recently developed in vitro cell-free assembly (CFA) assay to determine the RNA packaging into the assembled core (8). Each of the BTV inner core proteins was translated sequentially in an in vitro translation system together with ten full-length +ve sense single-stranded ssRNA segments. To avoid any interference between protein translation and ssRNA packaging, only the coding region of each BTV transcript was used for translation assay while uncapped full-length ssRNAs were used for packaging. In brief, VP1, VP4 and VP6, the proteins that form the polymerase complex, were first generated individually using S1, S4 and S9 segments respectively and then all 3 proteins were mixed and incubated with a set of ³²P-labelled T7-driven 10 full-length BTV transcripts. For each experiment either a set of the complete 10 ssRNA segments, or a set of 9 segments excluding one large (S2), one medium (S5) or one small (S10) ssRNA segment was used. The reaction mixture was then incubated sequentially with in vitro expressed VP3 to form the subcore and VP7 to form a stable core structure. The newly assembled cores were purified by a sucrose gradient centrifugation and the fraction containing cores (fraction 6, FIG. 1A (8)) was treated with RNase to remove unpackaged RNAs. The encapsidated RNAs in the cores were then phenol-chloroform extracted and analysed on a denaturing agarose gel. FIG. 1B shows that when all 10 RNA transcripts were present, a complete set of BTV RNAs were resistant to RNase treatment, indicating that cores were synthesised and RNA packaged. When segment S2 was excluded, packaging of all segments was decreased while still apparent (˜40% compared to full set), when S5 was excluded, packaging was significantly reduced (˜10%), but when S10 was omitted RNA packaging was abolished (undetectable on Phosphor-imager). The experiment was performed in triplicate with the same result, indicating that omission of different RNA segments has a variable influence on RNA packaging and that S10 plays a critical role in the packaging of BTV ssRNA segments.

To further examine and quantify the effect of exclusion of each RNA segment, we undertook a further set of packaging experiments. Except for S6, all segments were excluded individually from the set of non-radioactive 10 ssRNA segments in the CFA assay. In vitro assembled cores were purified and treated with RNase as before. An aliquot of each sample was stored for protein analysis and packaging was measured using quantitative RT-PCR (qRT-PCR) for a marker BTV ssRNA, S6. The S6 packaging efficiency from each set of 9 ssRNA segments packaging versus the complete 10 ssRNA set of control experiments was assessed. The qRT-PCR comparison results demonstrated that when S1, S2 or S3 was excluded, the packaging efficiency was ˜50%, while the exclusion of S4, S5 or S7, the medium size ssRNAs, reduced packaging further (15˜30%); but most strikingly, the packaging efficiency was as little as 10% or less when any of the smaller segments (S8, S9 and S10) were excluded (FIG. 1C). To confirm that protein expression in each experiment was equivalent, we determined the presence of polymerase complex protein VP6 and the major capsid protein VP7 by western blot analysis using mono-specific polyclonal antibody. All assembled samples, including controls and various segment exclusions, showed the presence of VP6 and VP7 proteins at similar levels, indicating that viral protein synthesis and core assembly was similar in all samples despite the absence of one segment (data not shown). The data confirmed a preferential role for smaller RNA segments in the initiation of BTV genome packaging.

### S10 UTRs Influence BTV RNA Packaging Both In Vitro and In Vivo.

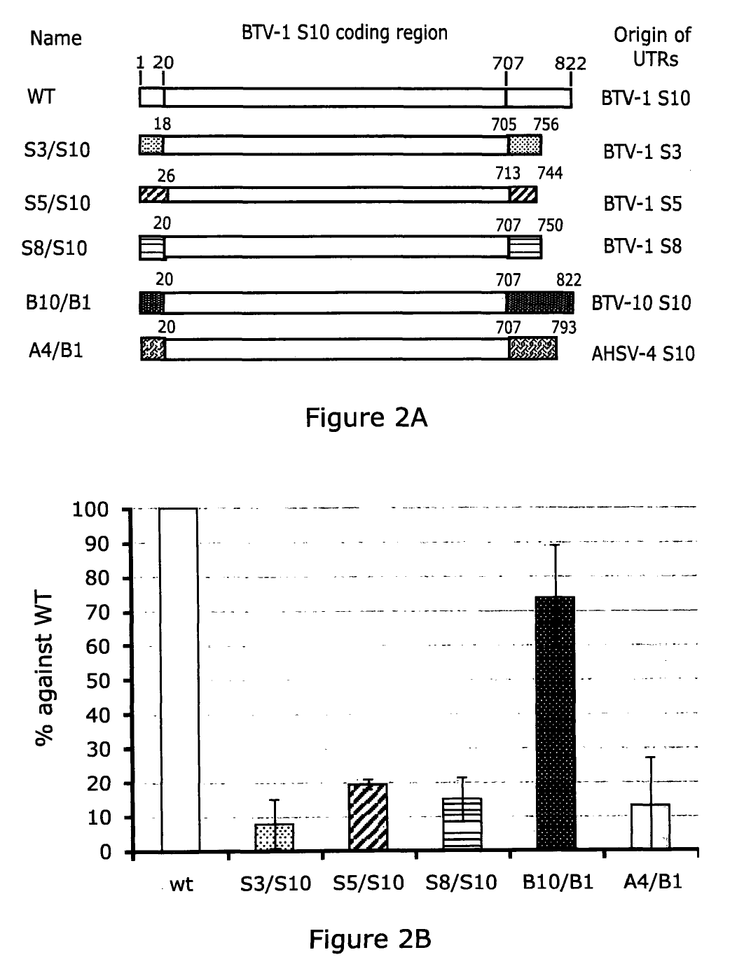

As S10 appears to play a critical role in BTV genome packaging, we investigated the relative roles of size and sequence identity. In many RNA viruses, specific packaging signals are mainly located in UTRs. Furthermore, among BTV RNA segments, although S10 is the smallest of all BTV RNA segments (822 bases), the 3′ UTR of S10 is unusually long (118 bases) when compared to the UTRs of other 9 RNA segments of BTV and is highly conserved among all serotypes. To verify if BTV S10 5′ and the long 3′ UTRs contain packaging signals, we designed chimeric ssRNA segments based on the coding region of BTV S10, with UTRs from different sources. To identify if S10 UTRs are essential for genomic RNA packaging, the UTRs of BTV-1 S10 were substituted with the UTRs of BTV-1 S3, S5, or S8, which are all different in both size and sequence. To verify the specificity of S10 UTR sequences, the UTRs of BTV-1 S10 were substituted with the UTRs of an alternate BTV serotype, BTV-10. The S10 of these two serotypes have similar but not identical sequences. In addition, S10 UTRs of a related orbivirus, African Horse Sickness Virus (AHSV), were also used to replace the UTRs of BTV-1 S10 (FIG. 2A). The sequence and predicted structural differences among these UTRs are shown in FIG. S1. Each chimeric construct was confirmed by sequencing and subsequently utilised to synthesise chimeric ssRNAs by in vitro T7 transcription assay.

To determine the effects of altered UTRs on RNA packaging, each chimeric S10 together with the remaining 9 BTV-1 ssRNA transcripts were used in the CFA system described above. In parallel, wild-type S10 transcripts were used as a positive control. The chimeric S10 transcripts that were packaged into the newly constituted cores were quantified with qRT-PCR and the packaging efficiency compared to that of the control. When the BTV-1 S10 UTRs were substituted with the UTRs of S3, S5, or S8 of the same serotype, the packaging was significantly reduced in each case, indicating that the UTRs of S10 were important for S10 incorporation into the core. Similarly, changing the S10 UTRs of BTV-1 to the UTRs of AHSV-4 also reduced packaging substantially. However, replacement with S10 UTRs of an alternate BTV serotype (BTV-10), to BTV-1 UTRs, influenced the packaging only moderately (˜70% efficiency, FIG. 2B). Thus, the packaging signals present in S10 UTRs are sequence-specific but closely related sequences from another BTV serotype could be tolerated.

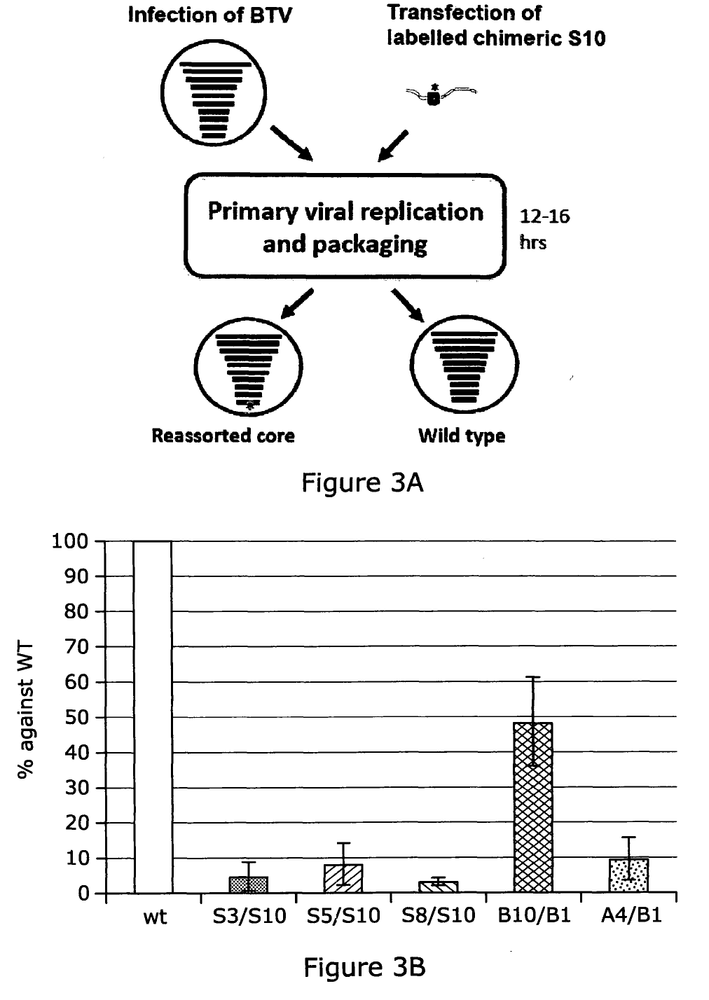

To determine if this effect can also be reproduced in vivo, we adapted a recently established in vivo single cycle packaging assay (19). The principle of this assay is that when BTV replicates in the cell, the progeny assembling cores will incorporate viral ssRNA segments from the cytoplasm. Therefore, if viral ssRNAs are transfected into cells prior to infection, both newly synthesised transcripts and transfected transcripts will be encapsidated. To perform the in vivo experiment, each chimeric S10 was introduced with a modified sequence in the coding region (nt 395, sufficiently distant to the UTRs) to facilitate specific detection and quantification by RT-PCR. This modification does not alter the amino acid sequence or the length of the segment (FIG. 3A). BSR cells were transfected with each modified chimeric S10 transcript or wild-type S10 T7 transcript followed by infection with BTV-1. After 12 to 16 hrs post infection, which allows for only one BTV replication cycle, transfected-infected cells were harvested and newly assembled viral cores were purified from the cell lysate as described (19). The modified S10 ssRNA packaged within the purified cores were then detected and quantified by qRT-PCR based on the specific modified sequence introduced in the S10 transcripts. To determine the packaging efficiency of the T7 ssRNAs, the copy number of the modified RNAs was correlated with the total number of transcripts present in the purified cores. The packaging efficiency of chimeric S10 was then compared with that of the control, wild-type S10 (FIG. 3B). The BTV-1/BTV-10 chimeric S10 ssRNAs were found packaged into new viral cores with an efficiency of ˜50%, as expected for a transcript competing with endogenously produced wild-type BTV-1 S10. However, all other chimeric S10 ssRNAs (S3/S10, S5/S10, and S8/S10 chimeras, and BTV-1/AHSV-4 chimeras) packaged very poorly, if at all. These in vivo data are consistent with the in vitro data obtained from the CFA assay and confirms that S10 UTRs are essential for BTV genome segment packaging and that the packaging signals concerned are highly specific and located in the UTRs.

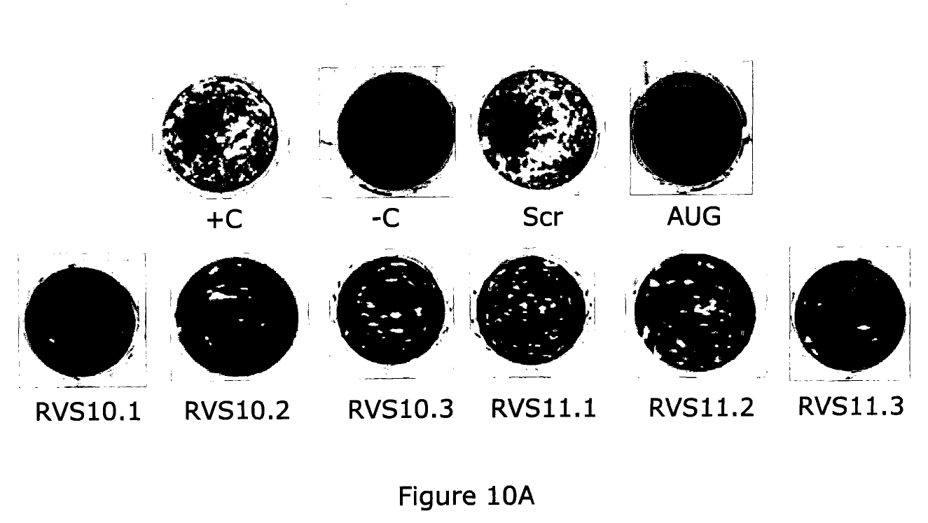

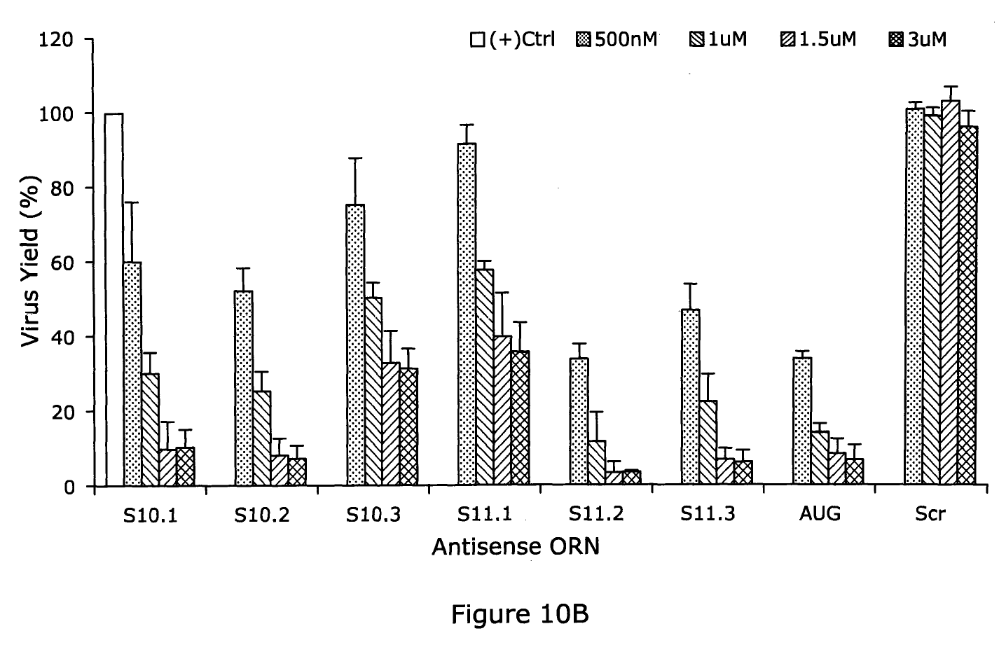

These effects were not restricted to BTV, but also confirmed in other members of the Reoviridae family (FIG. 10) wherein antisense targeting to the small segments of Rotavirus also drastically reduced virus yield.

### Changing S10 UTRs Blocked Viral Replication.

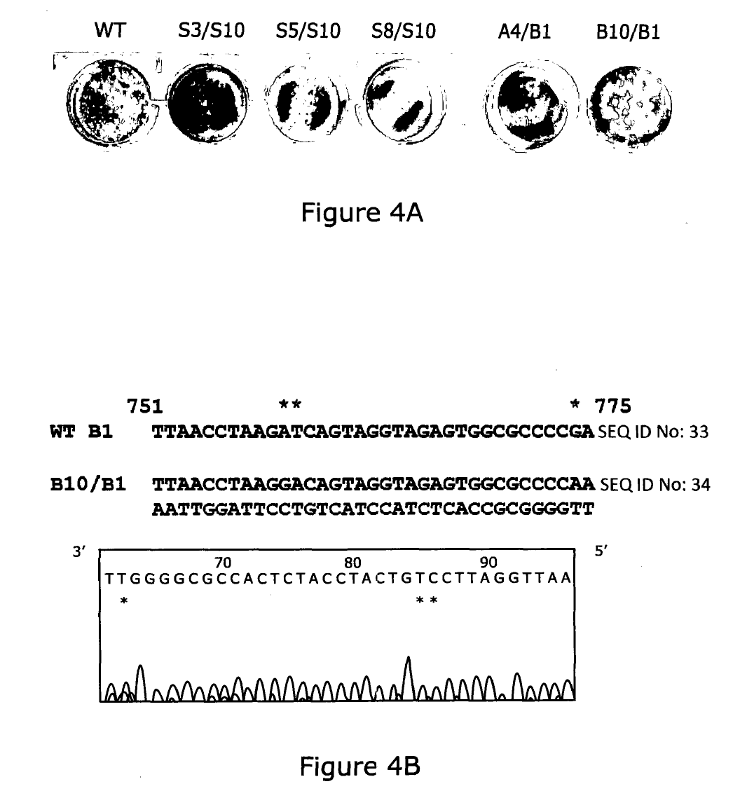

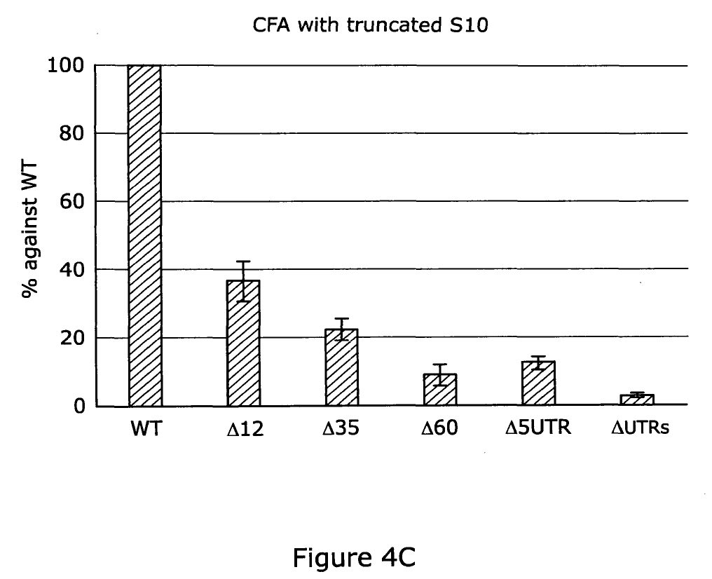

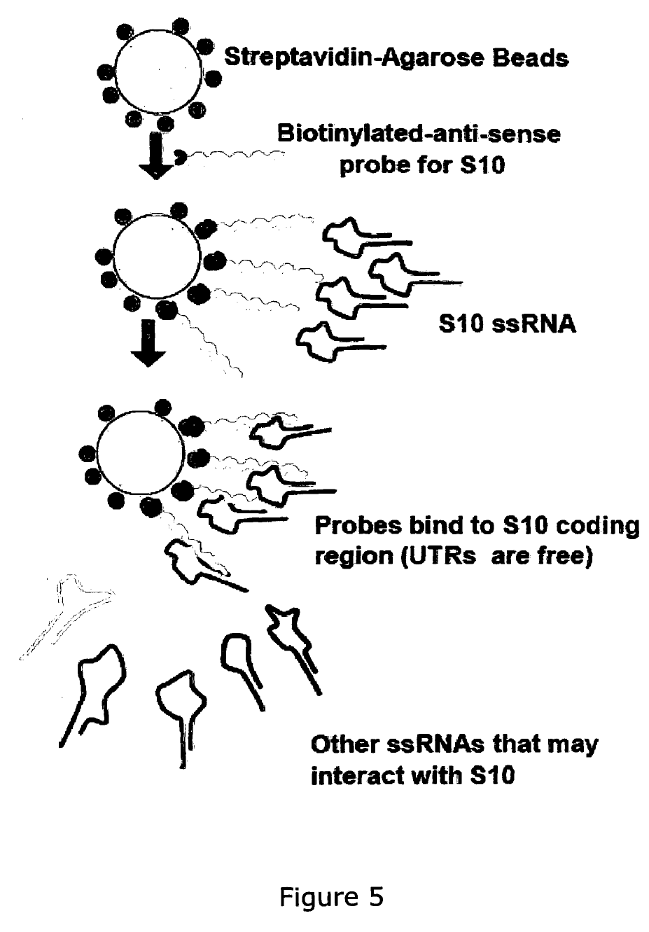

The above studies demonstrate that changing the UTRs of S10 influenced packaging both in vitro and in vivo. To further determine if poor levels of packaging can be compensated in the cellular environment, we verified the effect of the chimeric S10 constructs on viral replication using a BTV reverse genetic (RG) system which allows for the introduction of an altered genome segment in a replicating virus (17). Accordingly, the five chimeric S10 constructs described above were introduced together with the remaining 9 other wild-type BTV segments using the RG system in an attempt to recover mutant viruses. Among the five chimeric mutants, only BTV carrying BTV-1/BTV-10 chimeric S10 was successfully recovered, as examined by plaque morphology and titres, in comparison to that of the wild-type virus (FIG. 4A). That the recovered virus was not a revertant was confirmed by sequencing which showed the chimeric sequence to be present (FIG. 4B). No virus was detected when the other four chimeric S10 RNAs were used despite multiple experiments (N=3). Thus, changing the S10 UTRs by substituting with UTRs of other segments perturbs RNA packaging and effectively prevents viral replication. In contrast, when the S10 UTR is compatible with packaging, as in the BTV-1/BTV-10 exchange, packaging occurs and virus replication ensues.

Since S10 UTRs appear to be essential for BTV RNA packaging, we determined if certain specific region/regions in the UTRs are involved in RNA packaging. The 3′ terminal nucleotides of S10 was sequentially deleted from 12 to 60 nucleotides (12, 35 and 60) and each of these truncated S10 ssRNAs, together with remaining 9 full-length ssRNAs, were used for packaging in the CFA assay. When the packaging efficiency of each set was assessed, even the deletion of 12 nucleotides from the 3′ terminus suppressed packaging by more than 50%, and additional deletions further decreased packaging (FIG. 4C). The data suggests that the end of S10 3′ UTR plays a significant role in BTV genome packaging. When the entire 5′ UTR or both 3′ and 5′ UTR of S10 was deleted there was essentially no packaging of the remaining ssRNA segments. These data indicate that both termini of S10 are important for packaging, probably via their interaction with each other.

S10 Interacts with Other BTV RNA Segments.

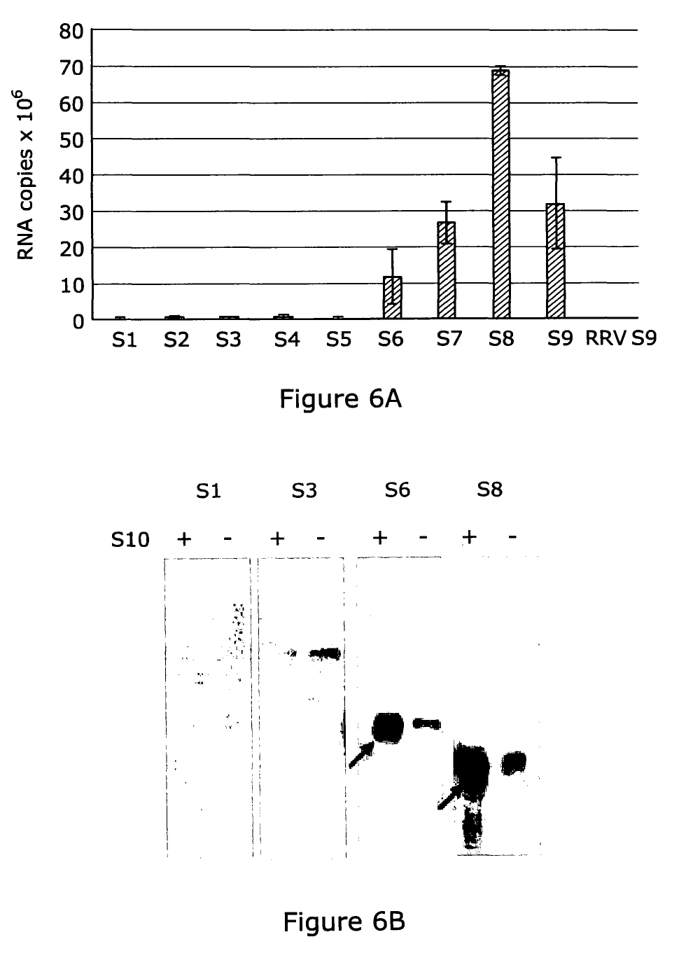

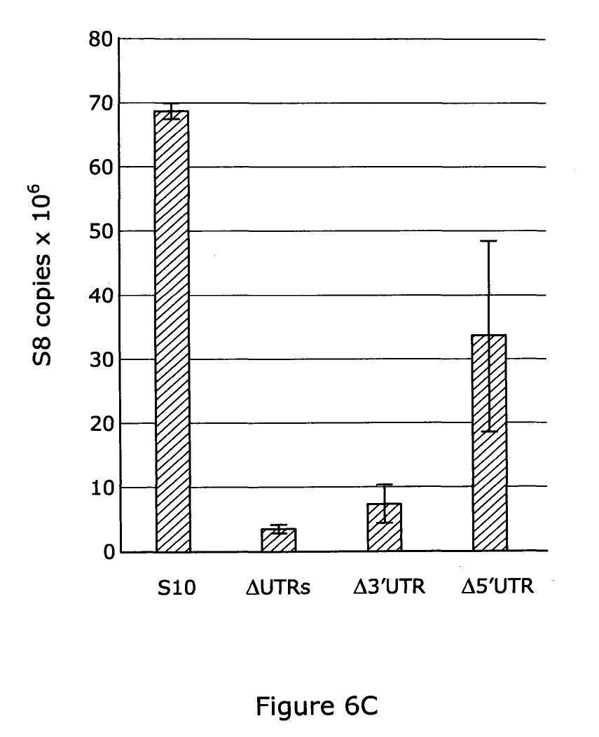

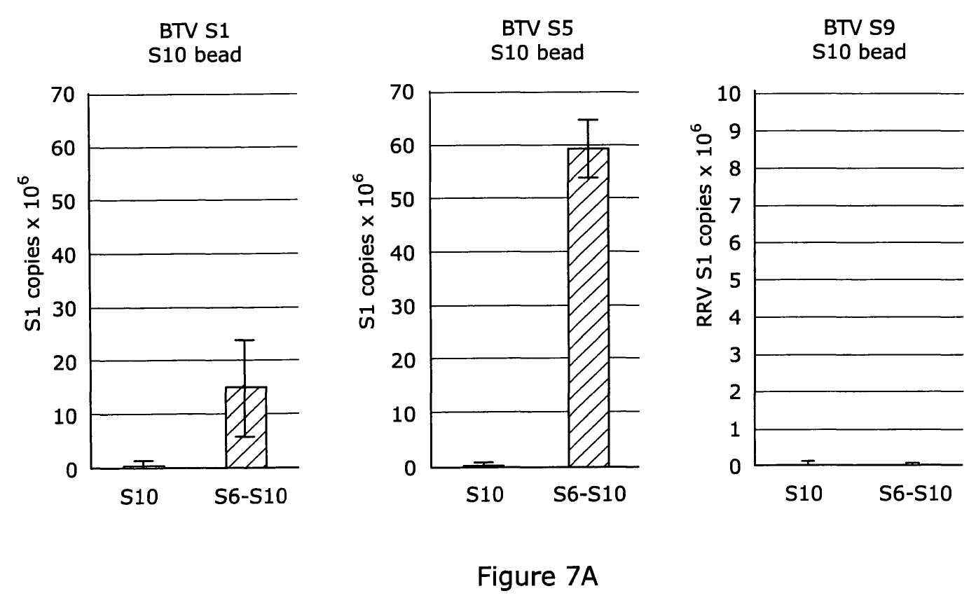

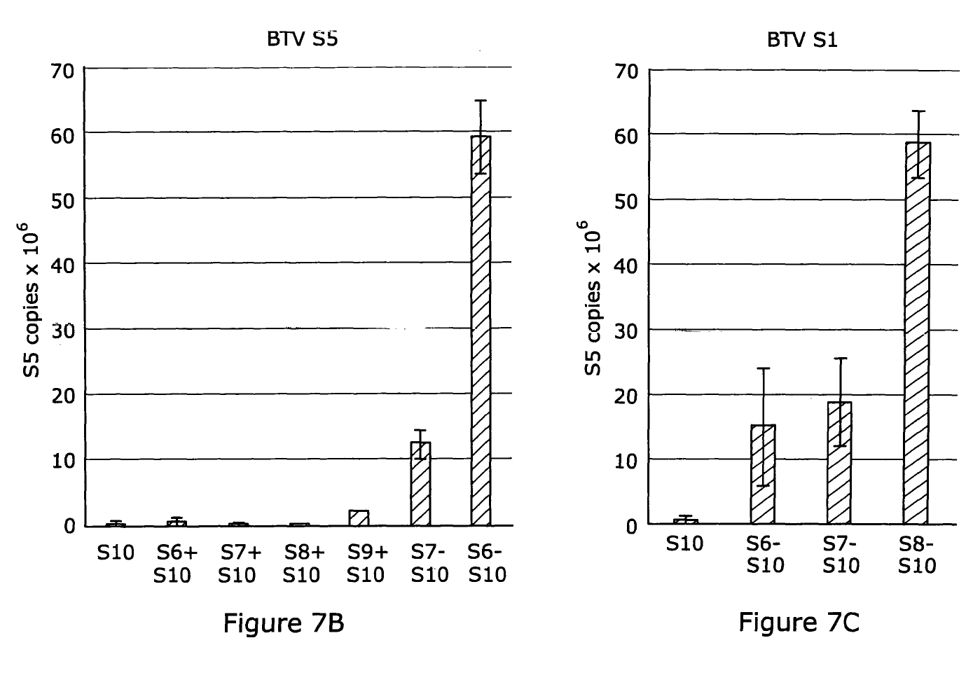

As the smallest BTV RNA segment, S10, appears to initiate the packaging of the remaining RNA segments, we investigated if S10 RNA recruits other segments by direct interaction. To detect interactions between different RNA segments, we designed a primer binding assay based on streptavidin beads as shown in a schematic (FIG. 6). Since the UTRs of S10 were important for assembly, it was necessary to keep both the 5′ and 3′ termini free, unbound to beads. A biotinylated primer which specifically binds to the centre of the S10 coding region was designed. The primer was used to coat the streptavidin beads and allowed to anneal to the S10 RNA. Then, different BTV RNA segments (BTV S1-S9 and ssRNA of a non-related Rhesus Rotavirus (RRV) RNA, S9), each at 1 pmole, were incubated with the coated beads. After washing, the attached RNA from each reaction was released and detected by qRT-PCR using segment-specific primers. Non-coated beads served as the negative control. The results indicated that S10 had a high affinity for the small BTV segments, S7, S8 and S9, particularly S8 and a moderate affinity for S6, a medium size RNA segment of BTV (FIG. 7A). S10 did not interact with the larger segments and showed essentially no affinity for RRV RNA S9 (814 bases). This suggests that the interaction between S10 and smaller BTV segments is not due to their size but is more likely sequence or secondary structure specific. To confirm these results, the same RNA-RNA pull-downs were also performed using radiolabelled ssRNAs. ³²P-labelled S1, S3, S6 and S8 were incubated separately with beads coated with unlabelled S10 RNAs as described above. Beads not coated with S10 were used as controls. After extensive washing, the bound ³²P-RNAs were released from the beads by heating at 90° C. and analysed on a denaturing agarose gel followed by autoradiography. It was clear that while both S6 and S8 had interacted with S10, the larger segments S1 and S3 failed to bind S10 (FIG. 7B). As the S10 UTRs are important for packaging, they plausibly also play a role in RNA-RNA interaction. To verify this, primer bound beads were coated with the coding region of S10, S10 with the 3′ UTR or S10 with the 5′ UTR only. Coated beads were incubated with S8, a representative segment shown to have the highest affinity for wild-type S10, and the binding was estimated. When both UTRs were removed, S10 largely lost its affinity for S8 and this was also the case when the 3′ UTR was removed. However, when the 20 bases of 5′ UTR were removed, S10 and S8 interacted to a level of ˜50% of the parental molecule (FIG. 7C). Thus, the 3′ UTR of S10 is critical for the observed S10-S8 interaction while the 5′ UTR is not essential but might enhance it.

### Smaller Segments Can Act as Intermediates for Binding the Larger Segments.

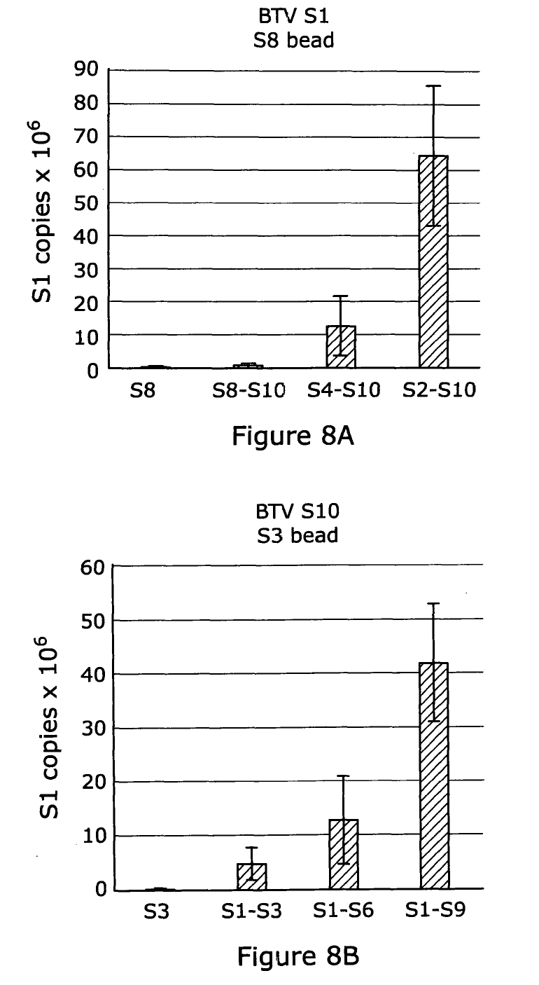

Isolated S10 exhibited an affinity in vitro for the smaller BTV segments but not the medium or large segments. However, for BTV genome packaging, all 10 segments have to be included to form a complete genome set. To enable this, the smaller segments plausibly form a complex which is then linked to other segments. To verify this hypothesis, we added S6, S7, S8 and S9 onto the S10 beads followed by incubation of the mixture with S1 or S5, as representatives of large and medium size segments respectively, each of which previously failed to bind to S10 directly. Clearly, in the presence of other small segments, both S1 and S5 were successfully pulled-down but not by S10 alone, while there was no change for the RRV RNA control (FIG. 8A). These data indicate that a complex might have formed with S6-S10, which was probably necessary to pull-down the larger segments.

Further, we investigated whether the increased affinity was mediated by one or more specific segments of the four RNA segments, S6-S9, or if all RNA segments formed a complex to recruit larger segments. When each of the small segments, S6 to S9 was added separately into the interaction assay of S10 with S5, only S9 slightly enhanced the interaction (FIG. 8B). However, a combination of three small segments (S7 to S10) significantly enhanced interaction with S5. Moreover, the presence of S6 in this mixture increased the affinity fivefold. None of the individual segments alone affected the S5 and S10 interaction, only a mixture of segments affected the binding of S5, suggesting that it is essential that smaller segments form a complex to bind to S5. When confirming this with S1, it was clear that the mixture containing S4-S9 had a similar effect to the one seen with the S6-S10 and the binding of S1 did not improve. However, when S2 and S3 were added to the mixture, the affinity increased threefold (FIG. 8C). These interactions suggest that there is an order of BTV genome RNA packaging, at least in the in vitro packaging assay, and that smaller segments form a complex which binds medium and then larger segments to effect full genome encapsidation.

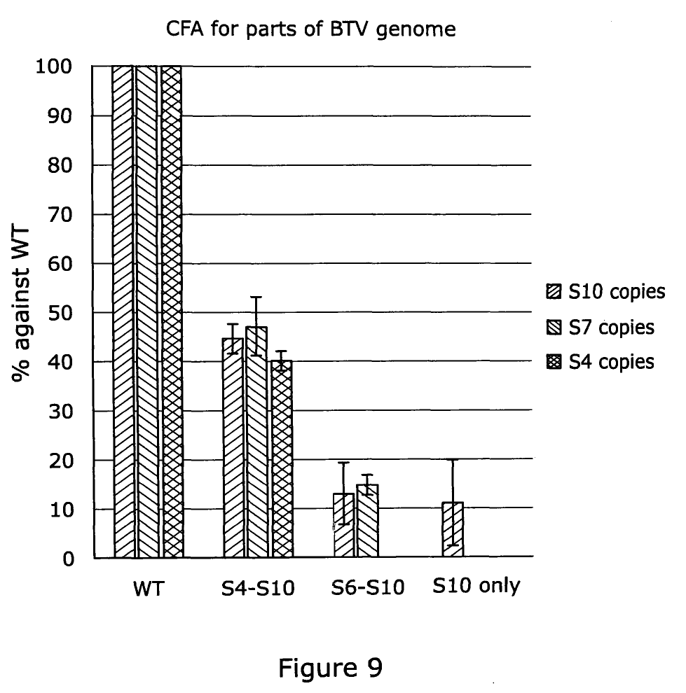

This was substantiated further by using an alternate smaller segment, S8, instead of S10, where the data obtained was similar (FIG. 9). There was no interaction when only S1 was added. Furthermore, S1 was not pulled-down by the complex formed between S8 and other smaller RNA segments (S9, S10). However, the mixture of S4-S10 interacted better with S1 and when S2 and S3 were added, the recruitment of S1 increased fivefold. In contrast, when S3 beads, instead of S8, were used to pull-down S10, there was no interaction and also significantly less when only large and medium size ssRNAs were first pulled-down by S3 beads. S10 was pulled-down most efficiently when all small segments were present as well.

### RNA-RNA Networking is Essential for Packaging.

The aforementioned data demonstrates that smaller segments are more important for BTV RNA packaging and that BTV RNA segments may form networks of size-related groups. Based on these, we hypothesised that such networking is important for BTV genome packaging. To demonstrate this, only certain genome segments were used in CFA system (FIG. 9). Results obtained showed that although S10 was previously shown to be important for BTV RNA packaging and containing packaging signals, S10 alone was not packaged in this in vitro assembly system. Moreover, when S6-S10 RNAs were used for packaging in the absence of larger segments, packaging was substantially reduced when compared to packaging of the full set of 10 segments. However, the addition of S4 and S5 in the S6-S10 mixture increased the packaging twofold to an equivalent of the packaging efficiency when only one of the large segments (S1, or S2, or S3) was excluded as shown in FIG. 1. In each packaging reaction, efficiency was determined by qRT-PCR analysis using three different segments (S4, S7 and S10) as necessary and all recorded similar incorporation. These results indicated that the packaging does not occur individually but rather depends on the correct complex formed by all RNA segments. Altogether the data suggests that the BTV genome was pre-assembled prior to being packaged into the capsid, and this assembly is likely based on a network of segments, which is initiated by the smaller segments.

### Oligonucleotides Targeting BTV RNA Segments Affect Virus Replication.

The data above showed that smaller size class RNA segments (S7-S10), in particular, S10, triggers a series of RNA-RNA interactions that initiate the recruitment and packaging of positive sense ssRNAs during BTV assembly. Based on these data, we sought to investigate whether small specific antisense oligoribonucleotides (ORNs) targeting the 3′ terminal sequences of these smaller segments would interfere with BTV growth. A set of oligonucleotides (ORNs) complementary to the UTRs of positive sense ssRNA segments, S9 and S10 were designed (FIG. 11). For stability and to avoid the cellular immune response, the 2′0H of the ribose of each ORN was modified to 2′O-methyl. The sequences of each ORN are presented in Table 2.

Six ORNs complementary to different regions including the 3′ conserved terminus of the S10 (FFIG. 1C) were designed to interfere with the RNA structures, and three of which encompass the entire length of the S10 3′ UTR. S10.1 was complementary to the extreme 41 nt (nt822-782) including the conserved sequence, S10.2 should base pair 39 nt from the stop codon towards the 3′ terminus (nt737-699) and the 34 nt of S10.5 complimentary to nt781-748, the gap between S10.1 and S10.2. The other ORNs targeted the structure outside of the 3′UTR; S10.3 to the terminal 35 nucleotides of the coding region (ORF), S10.4 in the ORF (nt595-561) and S10AUG, the initiation codon. For segment 9 (S9), the 3′ UTR consists of 44nts (nt1049-1006), and thus, three ORNs encompassed part of the UTR and part of the 3′ ORF (FIG. 11B). One ORN (S9.1) was complementary to the extreme 3′ terminal 33nt (nt1049-1017), while ORNs S9.2 and S9.3 were complementary to the last 40 nucleotides of the coding region including the stop codon (nt1005-966) or the middle section of the coding region (nt427-391), respectively. In addition, for positive controls, ORNs complementary to the 5′ UTR regions including the AUG codons of both S9 (S9 AUG) and S10 (S10 AUG) (FIG. 11, B & C; Table 2) and a SCR sequence of 30 nucleotides were also synthesized.

After optimizing the concentration of ORNs for in vivo assay, BSR cells were transfected with each ORNs and Scr ORNs at an optimal concentration of 1.5 μM. At 3 hours post transfection (hpt), cells were infected with BTV-1 of MOI of 0.1 and virus titres were monitored 16 hpi. Analysis of each ORN-transfected BSR cells followed by infection with BTV-1 showed ORNs had a negative effect on virus yield albeit to a varying degree with all S10 ORNs. Specifically, ORN S10.2 was the most inhibitory where virus yield was reduced by ˜90% while S10.3 had also a significant effect on virus replication with ˜70% reduction in comparison to that of the control (FIG. 11D). These ORNs were complementary to the 3′ end of the coding region (S10.3) and beginning of the 3′ UTR (S10.2). Secondary structure prediction of S10 revealed the S10.2 ORN was complementary to a GC rich hairpin loop, a bulge and a double-stranded region. S10.1 ORN, which covered the extreme 41 nts of 3′UTR, also had a significant inhibitory effect on virus yield (˜70% reduction). In contrast to these three ORNs, ORN S10.4, which targeted part of the coding region (nt595-561) was less inhibitory. That all S10 antisense ORNs had some interference activity on virus replication is consistent with the smallest BTV RNA segment playing a crucial role in virus replication, as reported (9). In contrast to S10, S9.1 ORN, complementary to the extreme 33 nt of S9 3′ UTR, had very little, if any, effect on virus recovery, only ˜6% reduction (FIG. 11D). However, virus growth was drastically reduced (˜80%) in the presence of S9.2, which encompasses the 40 terminal nucleotides (UTR+ORF) and to a lesser extent, S9.3 ORN (ORF only) with 50% virus yield. As expected, the presence of the control ORNs, S10AUG or S9AUG, severely reduced virus growth. On the contrary, parallel assays with scrambled sequences showed no inhibitory effect on BTV virus replication. Further, no cell toxicity was observed up to 48 hrs of incubation of BSR cells with different concentrations of Scr ORNs (0.1-2.5 μM) followed by staining the viable cells (data not shown), indicating that the effects of ORNs observed on BTV infected cells were specific to BTV replication.

Based on the inhibitory results of the ORN targeting the 3′UTR, we also investigated the effect of an ORN that encompasses an entire 3′UTR. We selected S1 as it possesses the shortest 3′UTR (24 nt) of all BTV RNA segments. To this end, we designed an ORN complementary to the entire length of the 3′UTR and, as positive control, another to the 5′UTR including the AUG codon (FIG. 11A). Virus titer was reduced to ˜80% in the presence of the S1 3′ ORN as compared to control without ORN and was similar to that of the 3′ UTR ORNs of S10 (FIG. 11D).

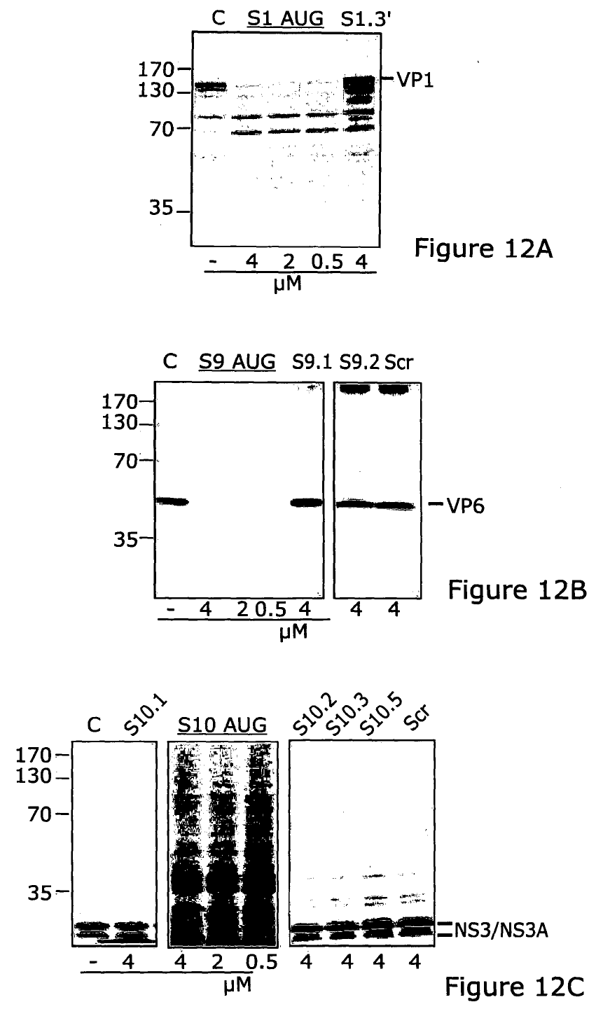

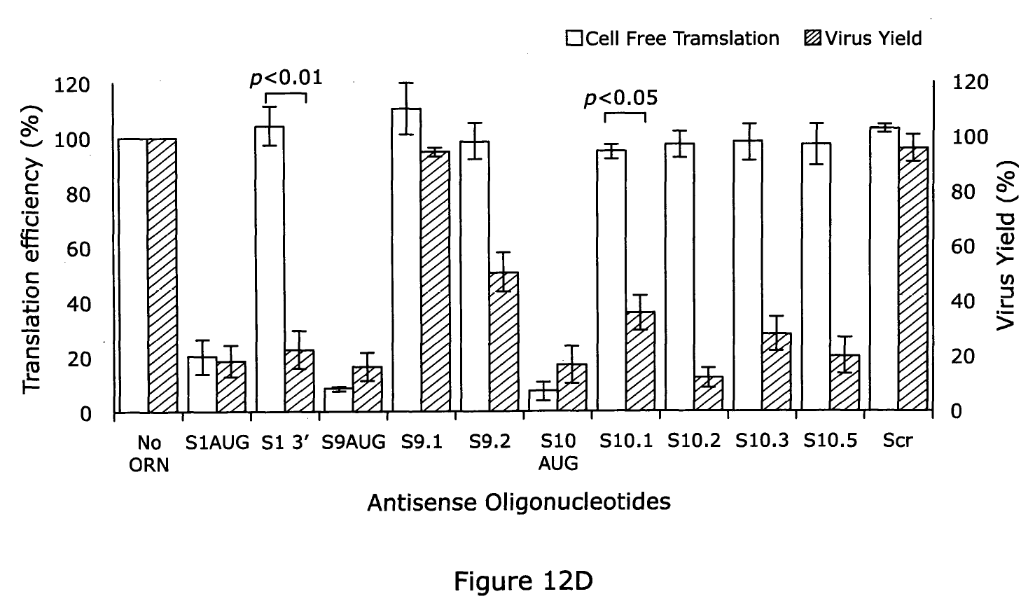

Since antisense oligonucleotides could trigger stearic blocking of viral mRNA and thereby perturb the translation of viral mRNAs, we examined if the inhibition of virus growth was due to the interfering effect of ORNs on the efficiency of virus protein expression. To validate this, we performed a cell-free translation in the presence or absence of ORNs complementary to the initiation codons of S1 (VP1), S9 (VP6) and S10 (NS3/NS3A) or the 3′ UTR region. Analysis of translated products showed that VP1, VP6, NS3/NS3a viral proteins were efficiently translated in the presence of ORNs complementary to the 3′UTR regions (FIGS. 12A-D). In contrast, a marked reduction of encoded protein levels were observed in the presence of S1, S9 and S10 AUG ORNs, respectively (FIG. 12A-D), consistent with the in vivo data (FIG. 12D). Conversely, scrambled control did not inhibit the translation of S9 and S10 mRNA (FIG. 12B, C and D), indicating sequence specificity of the ORNs to block their target regions. The significant inhibition of virus replication in the presence of 3′UTR ORNs in vivo in contrast to the efficient BTV protein synthesis in vitro suggests a mechanism of action whereby 3′UTRs of BTV RNA segments are important in virus replication.

Complex Networks of ssRNA Segments and Disruption by ORNs

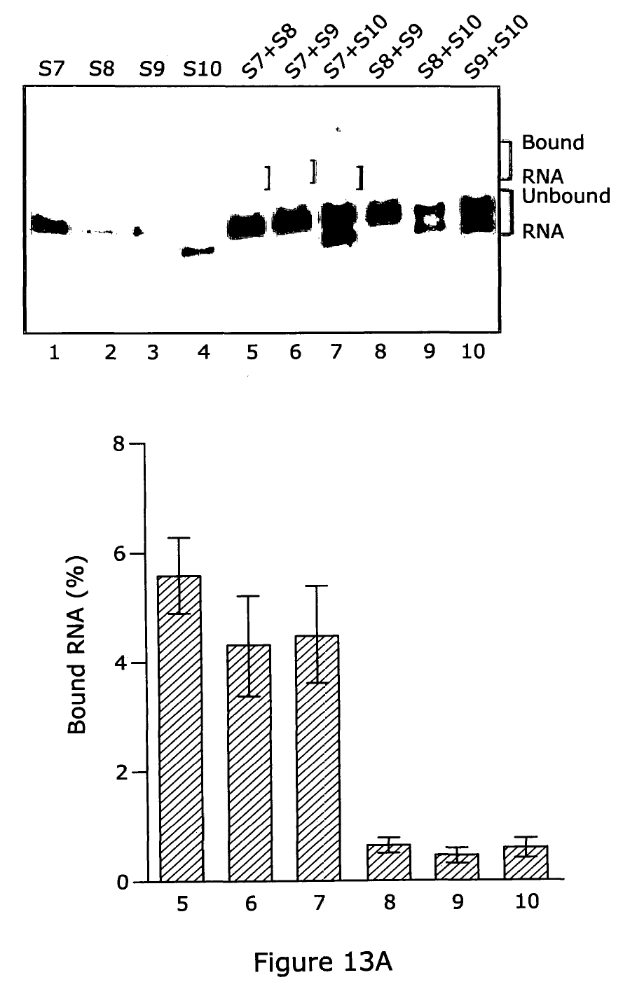

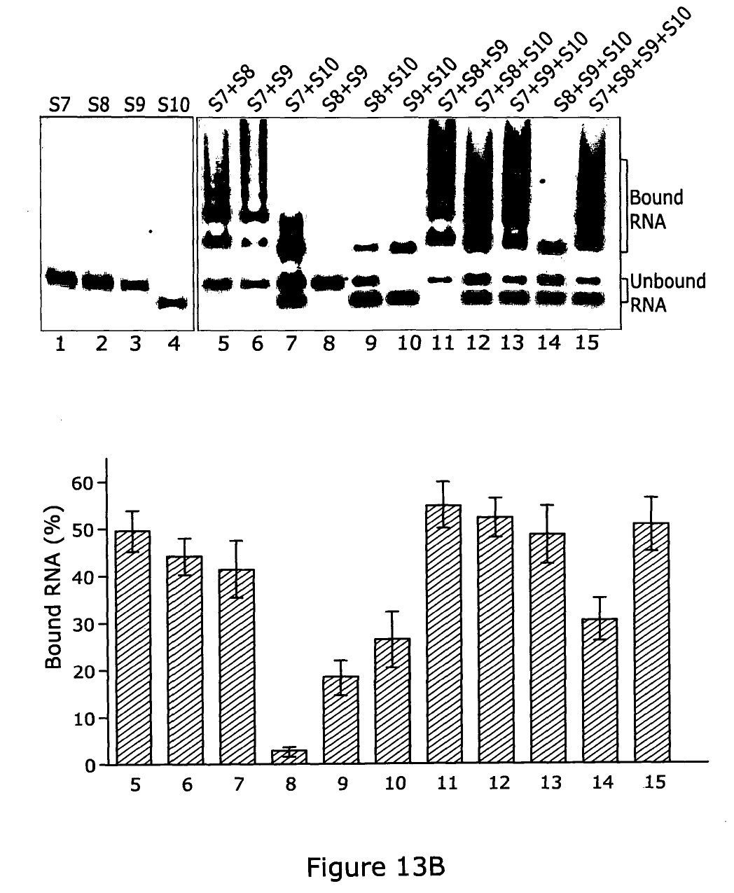

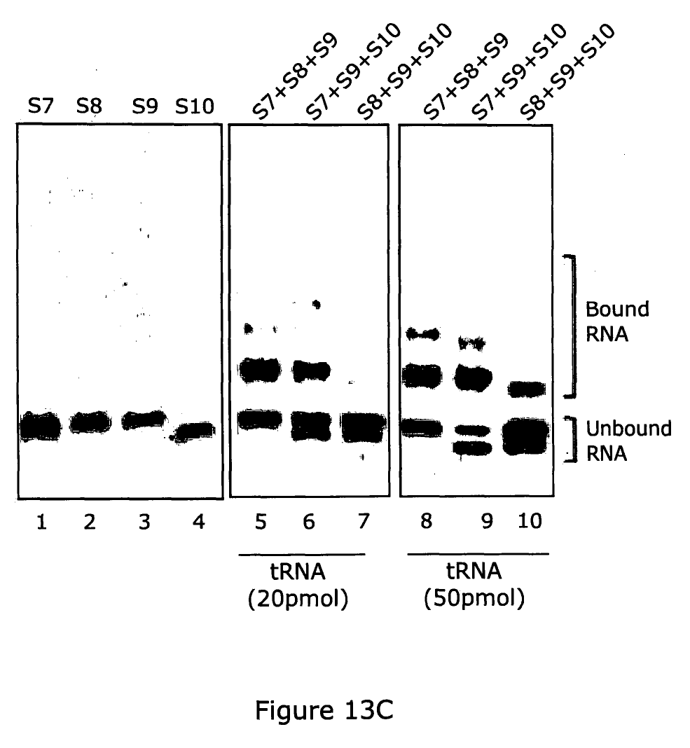

Previous RNA-RNA interaction data have shown that small size class RNA segments (S7-S10) interact with each other and package prior to the recruitment of medium and large RNA segments. To obtain direct evidence for interactions between smaller segments, we used electrophoretic mobility shift assay (EMSA) to measure RNA interaction following two different experimental approaches: (1) Co-incubation of two purified ssRNA segments for hybridisation assay and (2) Co-transcription of T7 cDNA copies of segments in pairs or in combinations of 3 or 4. The EMSA analysis of co-incubation products exhibited shifted weak bands for combinations S7+S8, S7+S9 and S7+S10 (FIG. 13A, lanes 5 to 7) indicating that

S7 interacts with each of the other three small segments. Other RNA segment combinations did not show any distinct retarded bands (FIG. 13A, lanes 8, 9, 10). In contrast to co-incubation, distinct retarded bands appeared when two segments were co-transcribed from T7 cDNAs (FIG. 13B, lanes 5 to 10), except S8+S9 (FIG. 13B, lane 8), suggesting that RNA segments were interacting during or soon after they were synthesized and the presence of S7 and S10 stimulated the complex formation.

In three or four co-transcribed RNA segments, stronger intermolecular interactions were detected with additional shifted bands each case and the amount of free, unbound RNA was also less than when only two segments were co-transcribed (FIG. 13). Further, the appearance of additional RNA complex were noticeable when S10 are present in the reaction (FIG. 13B, compare lanes 5, 6, 8 and 12 to 14) suggesting a key role for S10 in bringing the smaller segments together into a RNA network. The addition of S10 from a reaction with S7, S8 and S9 also led to stronger retarded bands (FIG. 13B, compare lanes 11 and 15) which strengthens the role of S10 in the intermolecular reaction. It is also evident that the presence of S7, which has the second longest 3′ UTR after S10, (FIG. 13B, compare lanes 8 to 10 and 11 to 13, also compare lanes 14 to 15) also appeared to be important for complex formation. Table 3 summarizes the results obtained from the RNA-RNA interaction studies of purified and co-transcribed segments.

The specificity of RNA-RNA interactions was tested in the presence of non-specific competitor yeast tRNA at 20-50-fold molar mass excess and the level of complex formation was not significantly reduced (FIG. 13C) indicating that interactions between RNA segments were sequence specific.

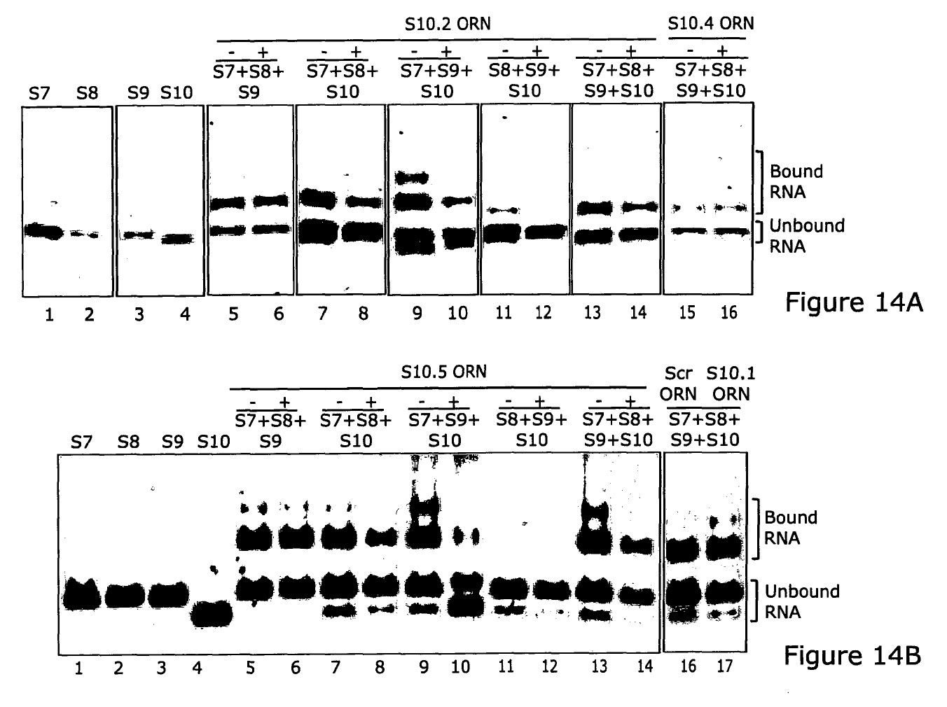

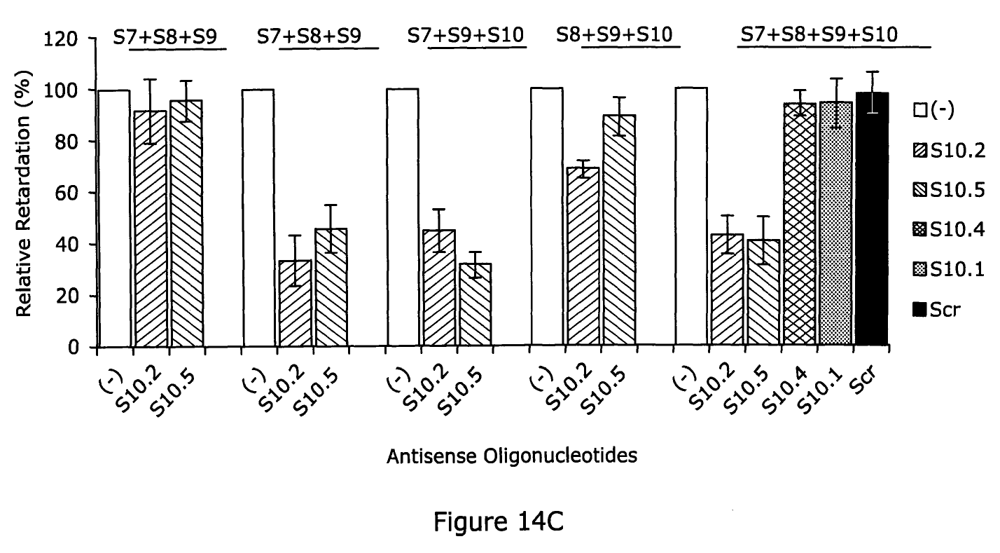

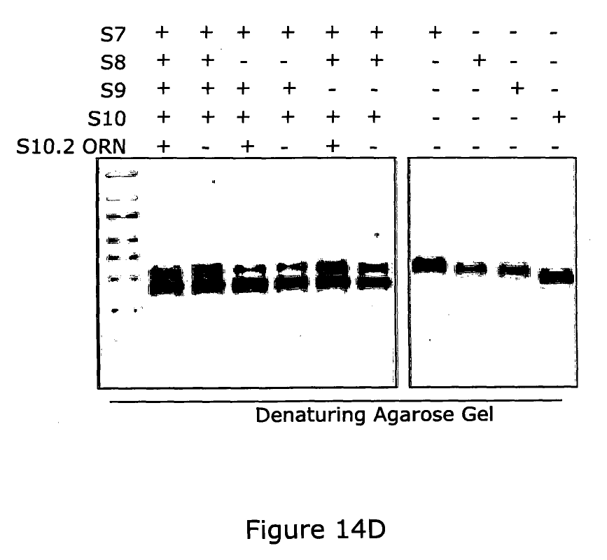

To determine if the RNA complexes following co-transcription of multiple segments could be disrupted by ORNs targeting the S10 3′UTR, all four small RNA segments or different combinations of three (S7+S8+S9, S7+S8+S10, S7+S9+S10, S8+S9+S10) were co-transcribed in the presence or absence of 20 pmol of either S10.2 and S10.5 ORNs (most inhibitory ORNs in virus replication) or S10.4 ORN (non-inhibitory ORNs targeting the ORF) (see FIG. 11, A, B, & C). EMSA data showed that RNA complexes in the presence of S10.2 and S10.5 were reduced up to four fold when compared to the control RNA complexes (FIG. 14, A and C) but not with S10.4. When the same reaction was undertaken in the absence of target RNA S10 (i.e.S7+S8+S9 only) the RNA complexes were not affected by the presence of S10.2 or S10.5 ORNs (FIG. 14 A and B, lanes 5-6).The RNA complex formed by S8, S9 and S10 in the presence or absence of S10.5 ORN was too weak to visualise whether S10.5 could inhibit the complex formation (FIG. 14B, lanes 11-12). These data suggest that the intermolecular interactions initiated by the S10 and S7 could be specifically disrupted by S10.2 (39nt) or S10.5 (34 nt) and emphasises that sequences encompassing by these two ORNs at the 3′UTR downstream of the S10 stop codon are involved in intermolecular RNA-RNA interaction. The S10.2 ORN was designed to target the GC rich hairpin loop, bulges and duplex while S10.5 targeted a duplex and hairpin loop. Results also suggested that the extreme 41 nt of S10 3′ UTR (S10.1) or the 35 nt in the S10 coding region (S10.4) appeared to be not essential for interactions.

The specificity of the ORN to inhibit RNA-RNA interactions was also shown by the non-inhibitory capacity of the Scr to disrupt the RNA complexes (FIG. 14B, lane 16), similar to the non-inhibitory results in virus replication and in vitro protein synthesis. The integrity of the transcribed RNAs was confirmed by denaturing gel analysis of the co-transcribed ssRNA segments which showed the position of the transcribed RNAs of each segment (FIG. 14D). The presence of distinct bands of complex and unbound RNAs as detected by native agarose gels showed that the RNAs were transcribed in these plasmid and ORN combinations.

Hybridization assay also showed that ORN S9 AUG and ORN S9.2 hybridized with S9 mRNA. Similarly, ORN S10 AUG and ORNs S10.2, S10.3, S10.5 annealed to S10 mRNA. No hybridization with Scr control was detected when incubated with S10 and S9 mRNAs.

Identification of Regions in S10 Responsible for Interactions with Other Segments

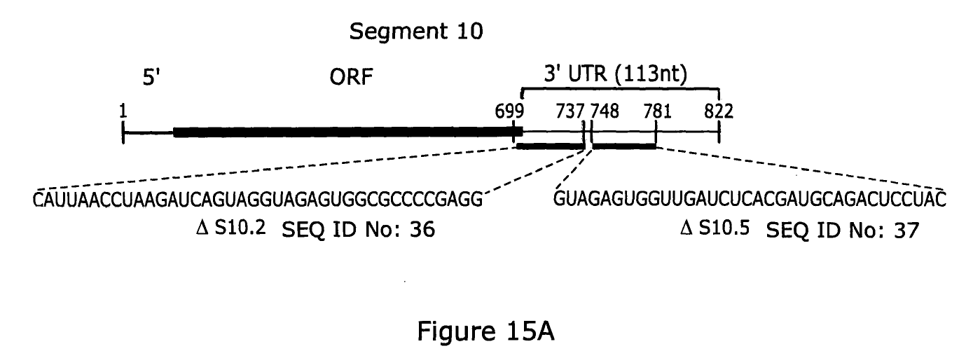

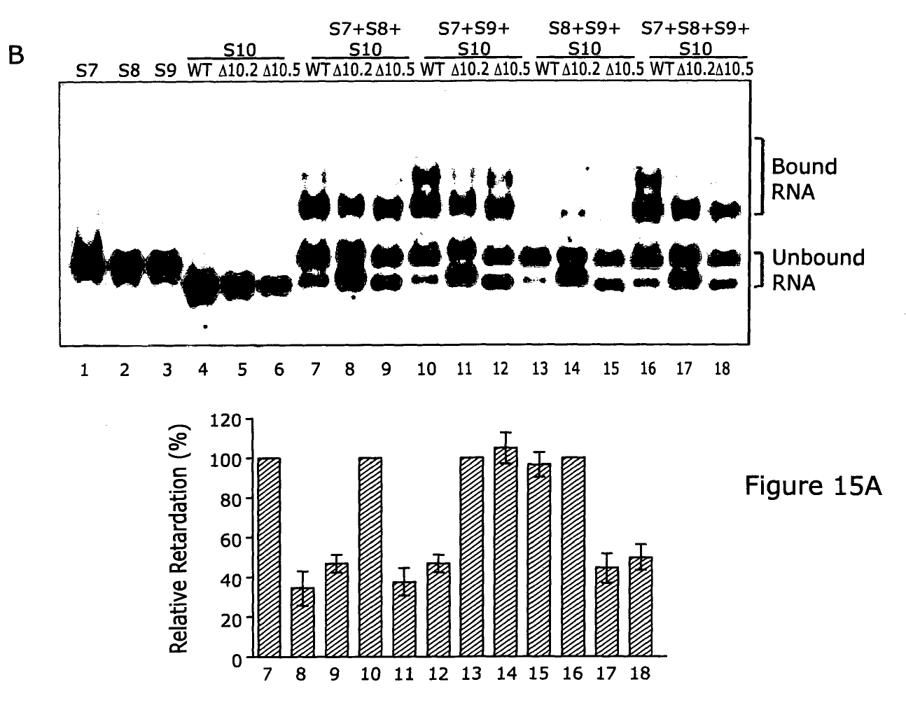

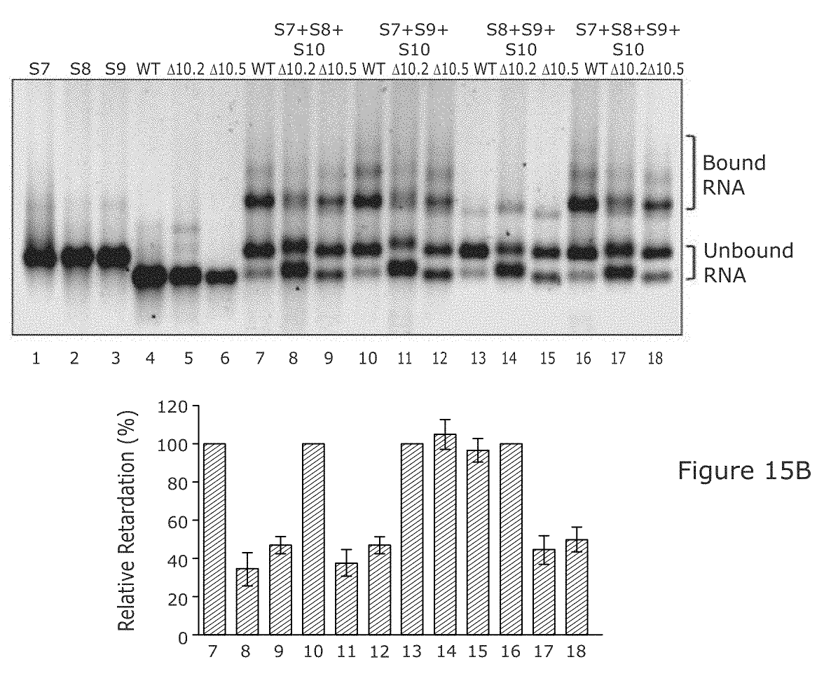

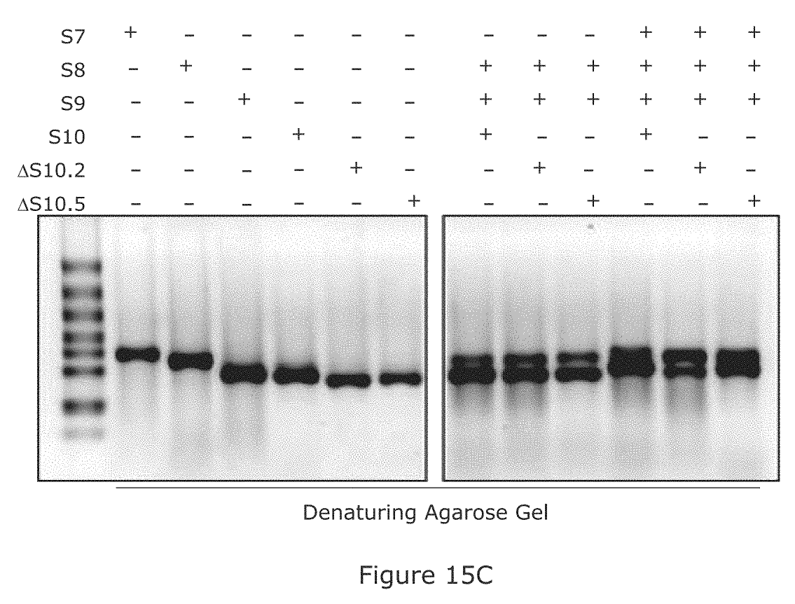

The decreased RNA complex formation in the presence of S10 3′UTR ORNs prompted us to explore the key regions in S10 RNA responsible for recruiting other segments to form a complex. Deletion mutants in S10 which spanned the sequence of inhibitory ORN binding region were constructed and used in the RNA-RNA interactions with other segments (FIG. 15 A). Up to four-fold reductions in RNA complex formation were observed with each of S10.2 and S10.5 deletion mutants in combination with S7+S8, S7+S9 and S7+S8+S9 when compared with the reactions with wild-type S10 (FIG. 15 B). No significant reduction of complex formation was observed with either of these S10 mutants when S8 and S9, but not S7, were present in accordance with the lower complex formation by these combinations. The RNA structures of deletion mutants showed that when target regions of S10.2 and S10.5 were deleted, the hairpin loops and bulges were either significantly altered or absent compared with the wild-type structure. This was consistent with the results obtained when using ORNs to inhibit RNA interactions (FIG. 14, A & B). The reduction of RNA complex formation in a reaction with deletion mutants S10.2 and S10.5 suggests the key role of S10 in recruiting other segments for complex formation and the importance of the sequence in the S10 3′UTR for intermolecular interactions which become more evident in the presence of S7 in segment combinations. The integrity of transcribed RNAs were confirmed by showing the position of the co-transcribed wild-type and mutant RNA segments on denaturing gel electrophoresis (FIG. 15C).

Table 4 summarizes the results obtained from RNA-RNA interaction studies in the presence or absence of ORNs and S10 deletion mutants.

### Specific ORN Inhibits BTV RNA Packaging During Cpsid Assembly

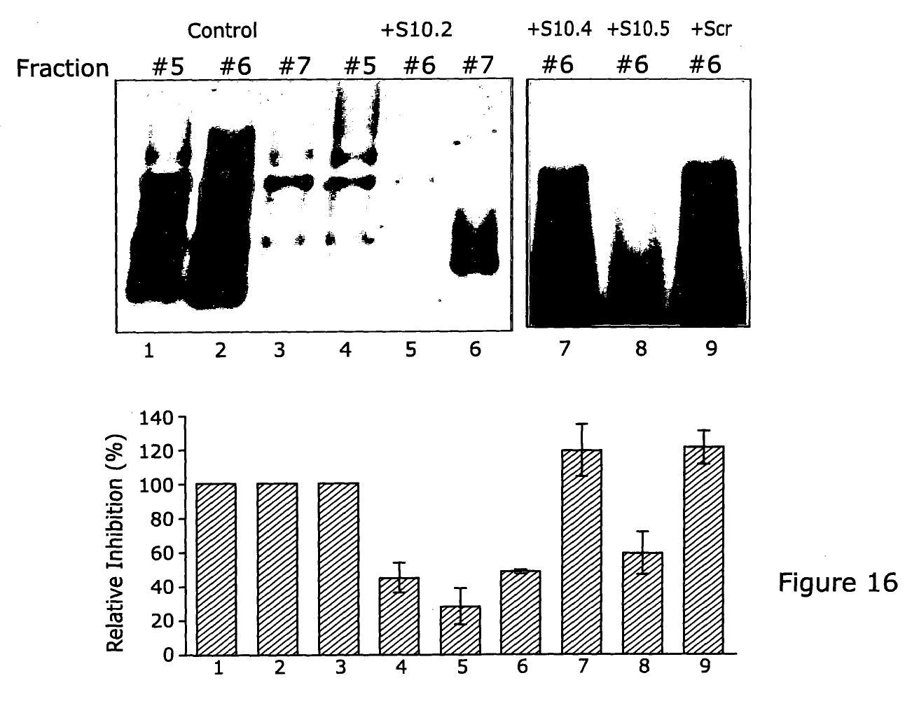

To further understand the mechanism of action of S10.2 and S10.5 ORNs and to determine if the inhibitory effects of ORNs on virus growth and RNA-RNA interactions were directly related to BTV RNA packaging during capsid assembly, we utilised a unique cell-free core assembly system for packaging BTV RNAs that has been successfully used to understand the order of capsid assembly and the genomic segment packaging. For this study, S10.1, S10.2, S10.5, S10.4, S10 AUG and Scr ORNs were pre-annealed to S10 transcripts prior to mixing with the remaining 9 BTV ssRNA segments and subsequently incubated with pre-translated transcription complex (VP1, VP4 and VP6) before adding two major core proteins, VP3 and VP7. After removing the unpackaged ssRNAs by RNase treatment, the putative cores in in vitro assembled complexes were centrifuged on a sucrose gradient, fractionated, ssRNAs isolated and analysed as described in Methods and Materials. Only S10.2 or S10.5 ORNs, (in fraction 6) inhibited the packaging of 10 BTV ssRNA with ˜80% reduction for S10.2 ORN and ˜60% reduction for S10.5 ORN (FIG. 16, lanes 4-6 and 8). The inhibition of packaged RNA was not noted on S10.4 and Scr ORNs (FIG. 16, lanes 7 & 9) and S10.1 and S10 AUG ORNs (data not shown). This indicates that by base pairing to its complementary sequence in the BTV genome, the ORNs S10.2 and S10.5 were capable of inhibition of RNA recruitment and packaging and possibly RNA-RNA interactions despite the presence of other nine BTV RNA segments.

To confirm that core proteins were synthesised efficiently in the cell-free assembly assay, each protein was ³⁵S-labeled and the fractionated complex was analyzed by SDS-PAGE. The ³⁵S-labelled reconstituted protein products showed the complete set of core proteins, the three proteins of transcription complex (VP1, VP4 and VP6) and the two major core proteins (VP3 and VP7) from fraction no. 6 in the presence or absence of S10.2 ORN (data not shown) which demonstrated that the transcription complex (TC) and the subcore proteins were efficiently synthesized and assembled and were not hindered in the presence of S10.2 ORN. The effects of different ORNs in RNA packaging by in vitro assembly, in vivo virus replication, in vitro protein synthesis and RNA-RNA interactions are summarized in Table 5.

### Virus Recovery is Inhibited by Substitution S10 Mutations and Chimeric 3′UTR

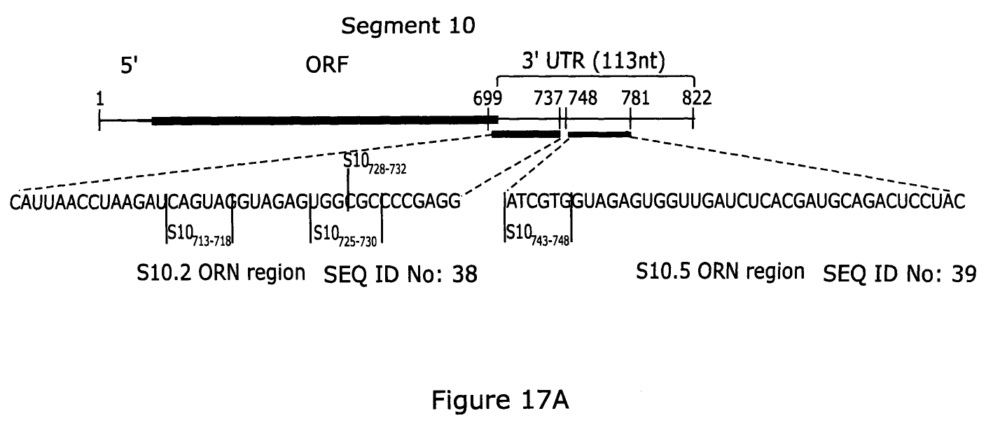

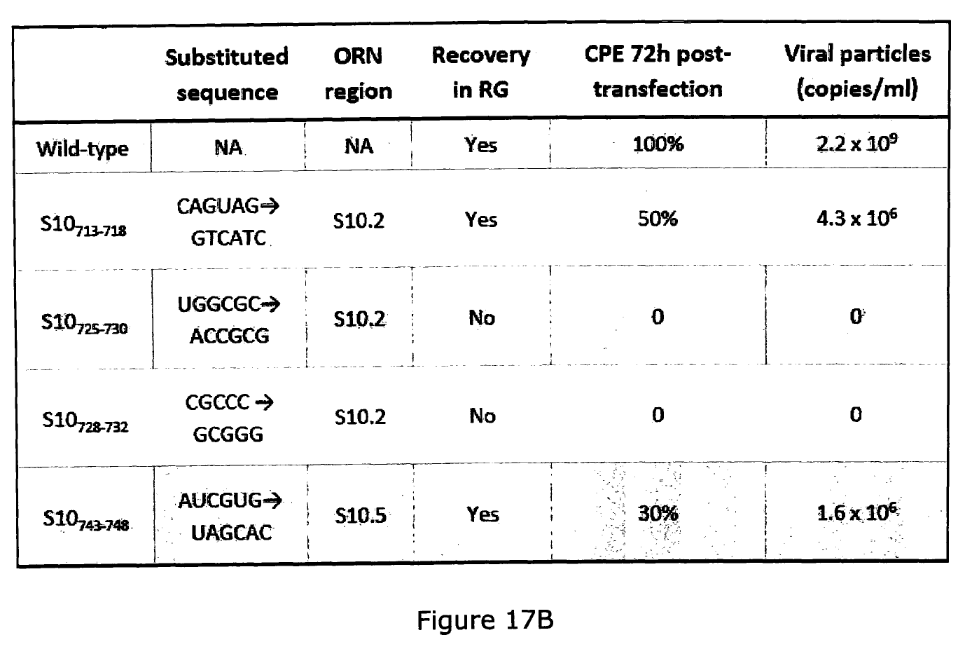

To confirm if the sequences within the identified 3′UTR regions in S10 RNA are important for RNA packaging in vivo, four substitution mutants were introduced by targeting five or six nucleotides in the putative binding sites of S10.2 and S10.5 regions at the S10 3′UTR (FIG. 17A). Each mutant S10 ssRNA was used to recover mutant viruses using reverse genetic system as described Materials & Methods. Among the mutants tested, only S10₇₁₃₋₇₁₈ (sequence covered by ORN S10.2) and S10₇₄₃₋₇₄₈ (sequence encompassed by ORN S10.5) were successfully recovered but exhibited significantly less cytopathic effects (CPE) and ˜1000 fold less viral particles compared to the wild-type at 72 hours post-transfection (FIG. 17B). The nucleotide substitutions in these two mutants were located in the double stranded region of the stem loop structure (S5). Mutants S¹⁰₇₂₅₋₇₃₀ and S¹⁰₇₂₈₋₇₃₂, which encompasses the hairpin loop of the S10.2 region, could not be rescued, consistent with a lethal phenotype.

To investigate further if the identified packaging signals in S10 3′UTR are interchangeable with other segments, 3′ UTRs of S8 and S10 were exchanged (S8-UTR10 and S10-UTR8) and chimeric ssRNAs were synthesized. When BSR cells were transfected with each of the chimeric RNA segments together with 9 WT ssRNA segments or all 10 WT ssRNAs as control, only control VVT virus was recovered while both chimeric segments failed in virus recovery. Further, virus recovery with combined S8-UTR10 and S10-UTR8 was unsuccessful. These data suggest that the packaging signals in the UTRs were not functional when interchanged between different segments.

### DISCUSSION

For the Reoviridae members with a multiple dsRNA segmented genome and complex capsid assembly process, understanding viral packaging, and so replication or viral yield, is highly challenging process. The data described in this report shows that members of the Reoviridae family assemble ssRNA segments of the viral genome through a selective and sequential process prior to packaging same as an RNA complex, moreover, this assembly is orchestrated by the smallest segment (e.g. S10 in BTV).

The core of BTV and other members of the family is a highly rigid icosahedral structure with 12 pores, one in each fivefold vertex, through which the newly synthesised viral positive strand ssRNAs extrude. Our recent data has shown that transcripts of each segment extrude through a specific pore and that it is not a random process. For the reverse process, i.e. the packaging of ssRNA through these narrow channels, RNA segment entry would have to occur one at a time, making it difficult to explain how excluding a single segment influences the packaging of other segments.

Our data showed that ORNs targeting the 3′ UTRs of the small segments (e.g. S9 and S10) had strong inhibitory effects on virus growth but not on protein synthesis suggesting that the inhibition occurred after viral protein synthesis and prior to genome encapsidation, at the stage of genome packaging.

Our data strongly suggests that the smallest segment, especially its UTRs, play a crucial function in RNA packaging and it is likely that interaction of the 5′ and 3′ UTRs drive the formation of secondary structure of ssRNAs, necessary for recruiting other ssRNAs.

However, other small segments (S7, S8 and S9) also interact with the smallest segment (S10) to trigger the assembly of all (10) ssRNAs. The data rationalises this finding by showing that the smaller segments form a complex, which then recruits the other segments. Interestingly, although the largest segment, S1, was captured by the small segment complex the interaction was enhanced significantly when large segments S2 and S3 were added. This data suggests that the 10 BTV segments may form several complexes which combine to result in a form compatible for packaging. Moreover, although S10 is crucial to BTV RNA packaging, neither S10 alone nor S10 plus other smaller segments were packaged efficiently. Only when larger segments were included were all segments equally packaged. This is consistent with an “all-in” genome incorporation model despite the fact that RNA-RNA interactions adopt a “follow-the-leader” model to assemble the packaging complex.

Our data indicates that the UTRs of S10 are critical for BTV assembly through a sequence-specific or secondary structure-specific mechanism. Even a short deletion (12 NT) from the 3′ terminus of S10 perturbed the packaging of the ssRNAs during assembly. Alignment of the different BTV serotypes shows a high level (over 80%) of conservation in the unusually long S10 UTR (shown in Table 1). It is possible that S10 interacts with other segments in some of these regions, which is consistent with the model that RNA secondary structure serves as the genome packaging signal for segmented viruses.

The complex formation through specific RNA-RNA interactions and the critical role of Small Segment 3′ UTRs were also substantiated by the reduction of RNA complex formation in the presence of ORNs (S10.2 and S10.5), which blocked critical motifs in the 3′ UTR of BTV S10, in particular, the predicted structure that consisted of GC rich hairpin loops and bulges. Blocking these motifs with ORNs affected the binding capacity of S10 for the other interacting RNA segments which was further confirmed by deleting regions corresponding to S10.2 and S10.5 ORN binding regions. Reduction in RNA complexes detected with deletion mutants AS10.2 and AS10.5 suggested that either the deleted sequences may form a part of the binding site necessary for RNA-RNA interactions or the deletions might have disturbed the secondary structure in these regions.

Our work suggests a critical role for the untranslated region of viral small segments in the virus life cycle, likely at the packaging and assembly level. This also suggests that the key interacting sequences in small segments are located at the 3′UTR of RNA segments.

### REFERENCES

9. Sung P Y, Roy P. Sequential packaging of RNA genomic segments during the assembly of Bluetongue virus. Nucleic Acids Res. 2014.

10. Lourenco, S. and Roy, P. (2011) In vitro reconstitution of Bluetongue virus infectious cores. Proceedings of the National Academy of Sciences of the United States of America, 108, 13746-13751.

12. Boyce M, Celma C C, Roy P. Development of reverse genetics systems for bluetongue virus: recovery of infectious virus from synthetic RNA transcripts. J Virol. 2008;82(17):8339-48

17. Boyce, M., Celma, C. C. and Roy, P. (2008) Development of reverse genetics systems for bluetongue virus: recovery of infectious virus from synthetic RNA transcripts. Journal of virology, 82, 8339-8348.

18. Toussaint, J. F., Sailleau, C., Breard, E., Zientara, S. and De Clercq, K. (2007) Bluetongue virus detection by two real-time RT-qPCRs targeting two different genomic segments. Journal of virological methods, 140, 115-123.

19. Burkhardt, C., Sung, P. Y., Celma, C. C. and Roy, P. (2014) Structural constraints in the packaging of bluetongue virus genomic segments. The Journal of general virology, 95, 2240-2250.

20. Fernandez-Miragall, O. and Martinez-Salas, E. (2003) Structural organization of a viral IRES depends on the integrity of the GNRA motif. Rna, 9, 1333-1344.

37. Weiner M P, Costa G L, Schoettlin W, Cline J, Mathur E, Bauer J C. Site-directed mutagenesis of double-stranded DNA by the polymerase chain reaction. Gene. 1994;151(1-2):119-23.

38. Qiao, X., Casini, G., Qiao, J. and Mindich, L. (1995) In vitro packaging of individual genomic segments of bacteriophage phi 6 RNA: serial dependence relationships. Journal of virology, 69, 2926-2931.

39. Ramos R, Martinez-Salas E. Long-range RNA interactions between structural domains of the aphthovirus internal ribosome entry site (IRES). RNA. 1999;5(10):1374-83.

40. Matsuo E, Roy P. Bluetongue virus VP6 acts early in the replication cycle and can form the basis of chimeric virus formation. J Virol. 2009;83(17):8842-8.

TABLE 1

| Virus/segment | 5′ UTR | 3′ UTR |
|---|---|---|
| BTV-1 S10 | GTTAAAAAGTGTCGC | GATCAGTAGGTAGAGTGGCGCCCCGAGGTCTGCATCGTGTAGAGTGGTTGAT |
| | TGCC | CTCACGATGCAGACTCCTACTGCTGTCTAACGGGGGAGGGTATGCGGCGCTA |
| | (SEQ ID No: 9) | CACACTTAC (SEQ ID No: 10) |
| BTV-10 S10 | GTTAAAAAGTGTCGC | GGACAGTAGGTAGAGTGGCGCCCCAAGGTTTACGTCGTGCAGGGTGGTTGAC |
| | TGCC | CTCGCGGCGTAAATTCCCACTGCTGTATAACGGGGGAGGGTGCGCGATACTA |
| | (SEQ ID No: 9) | CACACTTAC (SEQ ID No: 11) |
| AHSV-4 S10 | GTTAAAATTATCCCT | TATGACCTCCACGAGCGGAAAATCCATCGTGTTGGATGGATGGAACGCCTAG |
| | TGTC | ATCGTTTTCTAGGGAGTGGGATAACAACTTAC (SEQ ID No: 13) |
| | (SEQ ID No: 12) | |
| Rotavirus C | GGCTTTAAATTTTTC | AATCCCTGCGCTTCCTGCTGGTGAACGGACGCCATCCCGTTCATTTCTAGCG |
| (Bristol UK) | AGATCACTTTGCTCT | AGTAGAGAAAAACATTGTACCCGAAACGCTGAGTTGAGGATCAATGTAGATA |
| S11 | ACGAAGTA | TGAAAAATTCATGTGGCT (SEQ ID No: 15) |
| | (SEQ ID No: 14) | |
| Colorado | CACATTTTGTCTCTG | AGAGTGACCCTGGAGCCGTGCCGCATCTTCAATTTGTTAACAATGAGTTGAA |
| tick fever | TGATCCCCGCACAGA | GGTCGGGTGGAAGGTCGCGCCGATACGTCGTGATGGGCGAAACTACTCCATT |
| S11 | CGTTCCACT | CGTCTCAATGCTCGTAATCCTCAGTTAGGCGGTGCTTTTACGATTGAGAGCG |
| | (SEQ ID No: 16) | GAGCCTTTAAGGTGTAGTGTGAACGGGGCTAAGGCCTGGATACAAATGCAGT |
| | | G (SEQ ID No: 17) |
| Colorado | GACATTTTGTCTCAG | ATTTTATGCGTGACGGGCAGGGTAGTCGCTGAGGACCTCGCCGGCTTACGGA |
| tick fever | AACG | ATGATGACGTCCTAGCATCATTCTCCCGGGACGGGTAATTGCAGTC |
| S12 | (SEQ ID NO: 18) | (SEQ ID No: 19) |

TABLE 2

| | | | |
|---|---|---|---|
| | Antisense sequence 5′-3′ | | Length |
| ORN | (2′O-methyl modified) | Binding region | nt |

Segment 1 (3944 nt)

| | | | |
|---|---|---|---|
| S1 AUG | ACCAUUGCAUUUUAAC (SEQ ID NO: 20) | S1 5′ UTR + start codon | 16 |
| | | nt 16-1 | |
| S1 3′ | GUAAGUGUAAUGCGGCGCGUGCUC (SEQ ID NO: 21) | S1 3′ UTR | 24 |
| | | nt 3944-3921 | |

Segment 9 (1049 nt)

| | | | |
|---|---|---|---|
| S9AUG | UGACAUAUGCGAUUUUUUAAC (SEQ ID NO: 22) | S9 5′ UTR + start codon | 21 |
| | | nt 21-1 | |
| S9.1 | GUAAGUGUAAAAUCGCCCUACGUCAAGAAGGUA | S9 extreme 3′ UTR | 33 |
| | (SEQ ID NO: 23) | nt 1049-1017 | |
| S9.2 | UUAGAGGUGAUCGAUCAAAUGCAGGAACUCCGUUUUCACA | S9 coding region (3′ term + | 40 |
| | (SEQ ID NO: 24) | stop codon) nt 1005-966 | |
| S9.3 | CUUCUGUUAGAACUACCCAUCUUCCUCCAUUCGCUCC | S9 coding region (5′ term) | 37 |
| | (SEQ ID NO: 25) | nt 427-391 | |

Segment 10 (822 nt)

| | | | |
|---|---|---|---|
| S10AUG | AUCAGCCCGGAUAGCAUGGCAGCGACACUUUUUAAC | S10 5′ UTR + start codon | 36 |
| | (SEQ ID NO: 26) | nt 36-1 | |
| S10.1 | GUAAGUGUGUAGCGCCGCAUACCCUCCCCCGUUAGACAGCA | S10 extreme 3′ UTR | 41 |
| | (SEQ ID NO: 27) | nt 822-782 | |
| S10.2 | CCUCGGGGCGCCACUCUACCUACUGAUCUUAGGUUAAUG | S10 stop codon to 3′ UTR | 39 |
| | (SEQ ID NO: 28) | nt 737-699 | |
| S10.3 | UUAGGUUAAUGGUAAUUCGAAACCAUCUAGCGGGA | S10 coding region (3′ term + | 35 |
| | (SEQ ID NO: 29) | stop codon) nt 709-675 | |
| S10.4 | AAUUUGCUGGUUCAAGCUUCUCUCGCUUUUUGCGC | S10 coding region (3′ term) | 35 |
| | (SEQ ID NO: 30) | nt 595-561 | |
| S10.5 | GUAGGAGUCUGCAUCGUGAGAUCAACCACUCUAC | S10 3′ UTR nt 748-781 | 34 |
| | (SEQ ID NO: 31) | | |
| Scrambled | UGCUAUUACCAUGCUACAGAUGUAAGUGAU | scrambled sequence | 30 |
| (SCR) | (SEQ ID NO: 32) | | |

TABLE 3
Summary of RNA-RNA interactions between segments
(% of bound RNA)

| | | | | Interactions |
|---|---|---|---|---|
| | Interactions | Interactions of | | of co- |
| Two RNA | of purified | co-transcribed | Three & Four | transcribed |
| segments | RNA | RNA | segments | RNA |
| S7 + S8 | 5.6 +/− 0.8 | 50 +/− 4 | S7 + S8 + S9 | 55 +/− 5 |
| S7 + S9 | 4.3 +/− 0.8 | 44 +/− 4 | S7 + S8 + S10 | 53 +/− 4 |
| S7 + S10 | 4.5 +/− 0.8 | 42 +/− 6 | S7 + S9 + S10 | 49 +/− 6 |
| S9 + S10 | 0.6 +/− 0.2 | 27 +/− 6 | S8 + S9 + S10 | 31 +/− 4 |
| S8 + S10 | 0.4 +/− 0.1 | 18 +/− 4 | S7 + S8 + | 51 +/− 5 |
| S8 + S9 | 0.4 +/− 0.1 | 3.0 +/− 0.9 | S9 + S10 | |

TABLE 4

| | | | | RNA-RNA interactions of segments with | | | |
|---|---|---|---|---|---|---|---|
| RNA-RNA interactions of segments + ORNs | | | | S10 WT or S10 deletion mutants | | | |
| (% of relative RNA retardation) | | | | (% of relative RNA retardation) | | | |
| BTV | No | +S10.2. | +S10.5 | BTV | WT | | |
| segments | ORN | ORN | ORN | segments | S10 | ΔS10.2 | ΔS10.5 |
| S7 + S8 + S9 | 100 | 92 +/− 12 | 96 +/− 8 | S7 + S8 + S9 | N/A | N/A | N/A |
| S7 + S8 + S10 | 100 | 33 +/− 10 | 46 +/− 9 | S7 + S8 + S10 | 100 | 34 +/− 8 | 47 +/− 5 |
| S7 + S9 + S10 | 100 | 45 +/− 8 | 31 +/− 5 | S7 + S9 + S10 | 100 | 37 +/− 9 | 47 +/− 5 |
| S8 + S9 + S10 | 100 | 68 +/− 3 | 89 +/− 15 | S8 + S9 + S10 | 100 | 105 +/− 9 | 96 +/− 6 |
| S7 + S8 + | 100 | 42 +/− 7 | 40 +/− 9 | S7 + S8 + | 100 | 44 +/− 7 | 50 +/− 6 |
| S9 + S10 | | | | S9 + S10 | | | |

TABLE 5
Inhibitory effects of ORNs

| | | | RNA-RNA | |
|---|---|---|---|---|
| | In vivo | | interactions | |
| | virus | In vitro | (4 RNA | In vitro RNA |
| ORN | replication | translation | segments) | packaging |
| S10.1 | − | − | − | − |
| S10.2 | + | − | + | + |
| S10.4 | − | − | − | − |
| S10.5 | + | − | + | + |
| S10 | + | + | − | − |
| AUG | | | | |
| Scr | − | − | − | − |

1. A pharmaceutical composition effective against a member of the Reoviridae virus family comprising:
at least one oligonucleotide complementary to an untranslated region (UTR) of a nucleic acid located, either 5′ or 3′, adjacent a coding region of at least one viral genome segment& that constitutes a viral genome; and
at least one pharmaceutically acceptable carrier.

2. The pharmaceutical composition according to claim 1 wherein said viral genome segment constitutes the smallest or one of the small (S) segment(s) within the viral genome and is selected from the group comprising S6, S7, S8, S9, S10, S11 and S12.

3. The pharmaceutical composition according to claim 2 wherein said viral genome segment is any one or more of S7-10 in Bluetongue virus (BTV); any one or more of S7-10 in African horse sickness virus (AHSV); any one or more of S6-11 in Rotavirus; and any one or more of S6-12 in Colorado Tick Virus.

4. The pharmaceutical composition according to claim 1, wherein said untranslated region of nucleic acid is located 3′ of said coding region.

5. The pharmaceutical composition according to claim 1, wherein said untranslated region of nucleic acid is located 5′ of said coding region.

6. The pharmaceutical composition according to claim 1, wherein said oligonucleotide comprises between 7-45 bases and has at least 5, 6, 7, 8, 9, 10, 11, 12, 13, 14, or 15 consecutive complementary bases having regard to the UTR to which the oligonucleotide.

7. The pharmaceutical composition according to claim 1, wherein said oligonucleotide is complementary to the whole or a part of the longest 3′ UTR of the small (S) segment(s) within the viral genome.

8. The pharmaceutical composition according to claim 1, wherein said oligonucleotide is modified.

9. The pharmaceutical composition according to claim 9 wherein said oligonucleotide is modified to replace 2′OH of each ribose with 2′O-methyl.

10. The pharmaceutical composition according to claim 1, wherein said oligonucleotide comprises a consecutive sequence of bases equal to the entire 5′ or 3′ UTR.

11. The pharmaceutical composition according to claim 1, wherein said oligonucleotide is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to the UTR with which it is complementary.

12. The pharmaceutical composition according to claim 1, wherein said Reoviridae virus is selected from the group comprising: Cardoreovirus, Mimoreovirus, Orbivirus, Phytoreovirus, Rotavirus, Seadornavirus, Aquareovirus, Coltivirus, Dinovernavirus, Idnoreovirus, Reovirus and Mycoreovirus.

13. The pharmaceutical composition according to claim 12 wherein said Reoviridae virus is selected from the group comprising: Colorado tick virus, Aquareviruses, fusogenic orthoreviruses, orbiviruses, African horse sickness virus, Bluetongue virus, Seadornavirus, Avian reovirus and Rice dwarf virus.

14. The pharmaceutical composition according to claim 1, wherein said pharmaceutical composition comprises a plurality of said oligonucleotides.

15. The pharmaceutical composition according to claim 14 wherein said oligonucleotides target both the 5′ and 3′ UTR of at least one viral genome segment.

16. The pharmaceutical composition according to claim 14, wherein said oligonucleotides target at least one UTR of a plurality of viral genome segments.

17. The pharmaceutical composition according to claim 16 wherein said oligonucleotides target both the 5′ and 3′ UTR of said plurality of viral genome segments.

18. The pharmaceutical composition according to claim 14, wherein said selected viral genome segment(s) is/are the smallest or at least one of the small (S) segments(s) in the viral genome.

19. The pharmaceutical composition according to claim 1, wherein said oligonucleotide is complementary to the whole or a part of a UTR selected from the group comprising: SEQ ID Nos: 9, 10 11, 12, 13, 14, 15, 16, 17, 18 and 19.

20. The pharmaceutical composition according to claim 1, wherein said oligonucleotide is selected form the group comprising:
(SEQ ID No: 22)
UGACAUAUGCGAUUUUUUAAC;
(SEQ ID No: 23)
GUAAGUGUAAAAUCGCCCUACGUCAAGAAGGUA;
(SEQ ID No: 24)
UUAGAGGUGAUCGAUCAAAUGCAGGAACUCCGUUUUCACA;
(SEQ ID No: 25)
CUUCUGUUAGAACUACCCAUCUUCCUCCAUUCGCUCC;
(SEQ ID No: 26)
AUCAGCCCGGAUAGCAUGGCAGCGACACUUUUUAAC;
(SEQ ID No: 27)
GUAAGUGUGUAGCGCCGCAUACCCTCCCCCGUUAGACAGCA;
(SEQ ID No: 28)
CCUCGGGGCGCCACUCUACCUACUGAUCUUAGGUUAAUG;
(SEQ ID No: 29)
UUAGGUUAAUGGUAAUUCGAAACCAUCUAGCGGGA;
(SEQ ID No: 30)
AAUUUGCUGGUUCAAGCUUCUCUCGCUUUUUGCGC;
(SEQ ID No: 31)
GTAGGAGTCTGCATCGTGAGATCAACCACTCTAC;
and
(SEQ ID No: 32)
UGCUAUUACCAUGCUACAGAUGUAAGUGAU.

21. The pharmaceutical composition according to claim 1 wherein said composition is formulated for oral, rectal, nasal, bronchial, topical, vaginal, or parenteral administration.

22. An inhaler comprising the pharmaceutical composition according to claim 1.

23. A method for preparing a pharmaceutical composition comprising bringing an oligonucleotide complementary to an untranslated region (UTR) of nucleic acid located, either 5′ or 3′, adjacent the coding region of at least one viral genome segments that constitutes the viral genome in conjunction or association with a pharmaceutically or veterinarily acceptable carrier or vehicle.

24. A combined pharmaceutical composition comprising the pharmaceutical composition according to claim 1 and one or more different additional anti-viral agents.

25. A method for treating a viral infection comprising administering to an individual an effective amount of the pharmaceutical composition according to claim 1.

26. The method according to claim 25 wherein said individual is a human or an animal.

27.-30. (canceled)


SEQUENCE LISTING (39 sequences)

SEQ ID NO: 1 - DNA, 16 bp, Bluetongue virus
gttgaaaagtgaccta

SEQ ID NO: 2 - DNA, 16 bp, Bluetongue virus
actaaagagcgatttg

SEQ ID NO: 3 - DNA, 22 bp, Bluetongue virus
gttgaaaagtgacctaggaggc

SEQ ID NO: 4 - DNA, 21 bp, Bluetongue virus
ttcaccacacctaacattggg

SEQ ID NO: 5 - DNA, 17 bp, Bluetongue virus
cccaaatcgctctttag

SEQ ID NO: 6 - DNA, 41 bp, Bluetongue virus
tttttttttttgtattatagctcttttcttctttaagcctc

SEQ ID NO: 7 - DNA, 45 bp, Bluetongue virus
ttttttttttgcttcatcatcatccagcgtgactcttcccttggc

SEQ ID NO: 8 - DNA, 49 bp, Bluetongue virus
ttttttttttcaacatctattgtagcccatccattatatcctgttcctg

SEQ ID NO: 9 - DNA, 19 bp, Bluetongue virus
gttaaaaagtgtcgctgcc

SEQ ID NO: 10 - DNA, 113 bp, Bluetongue virus
gatcagtaggtagagtggcgccccgaggtctgcatcgtgtagagtggttgatctcacgatgcagactcctactgctgtctaacgggggagggtatgcggcgctacacacttac

SEQ ID NO: 11 - DNA, 113 bp, Bluetongue virus
ggacagtaggtagagtggcgccccaaggtttacgtcgtgcagggtggttgacctcgcggcgtaaattcccactgctgtataacgggggagggtgcgcgatactacacacttac

SEQ ID NO: 12 - DNA, 19 bp, African horse sickness virus
gttaaaattatcccttgtc

SEQ ID NO: 13 - DNA, 84 bp, African horse sickness virus
tatgacctccacgagcggaaaatccatcgtgttggatggatggaacgcctagatcgttttctagggagtgggataacaacttac

SEQ ID NO: 14 - DNA, 38 bp, Rotavirus subgroup 1
ggctttaaatttttcagatcactttgctctacgaagta

SEQ ID NO: 15 - DNA, 122 bp, Rotavirus subgroup 1
aatccctgcgcttcctgctggtgaacggacgccatcccgttcatttctagcgagtagagaaaaacattgtacccgaaacgctgagttgaggatcaatgtagatatgaaaaattcatgtggct

SEQ ID NO: 16 - DNA, 39 bp, Colorado tick fever virus
cacattttgtctctgtgatccccgcacagacgttccact

SEQ ID NO: 17 - DNA, 209 bp, Colorado tick fever virus
agagtgaccctggagccgtgccgcatcttcaatttgttaacaatgagttgaaggtcgggtggaaggtcgcgccgatacgtcgtgatgggcgaaactactccattcgtctcaatgctcgtaatcctcagttaggcggtgcttttacgattgagagcggagcctttaaggtgtagtgtgaacggggctaaggcctggatacaaatgcagtg

SEQ ID NO: 18 - DNA, 19 bp, Colorado tick fever virus
gacattttgtctcagaacg

SEQ ID NO: 19 - DNA, 98 bp, Colorado tick fever virus
attttatgcgtgacgggcagggtagtcgctgaggacctcgccggcttacggaatgatgacgtcctagcatcattctcccgggacgggtaattgcagtc

SEQ ID NO: 20 - RNA, 16 nt, Bluetongue virus
accauugcauuuuaac

SEQ ID NO: 21 - RNA, 24 nt, Bluetongue virus
guaaguguaaugcggcgcgugcuc

SEQ ID NO: 22 - RNA, 21 nt, Bluetongue virus
ugacauaugcgauuuuuuaac

SEQ ID NO: 23 - RNA, 33 nt, Bluetongue virus
guaaguguaaaaucgcccuacgucaagaaggua

SEQ ID NO: 24 - RNA, 40 nt, Bluetongue virus
uuagaggugaucgaucaaaugcaggaacuccguuuucaca

SEQ ID NO: 25 - RNA, 37 nt, Bluetongue virus
cuucuguuagaacuacccaucuuccuccauucgcucc

SEQ ID NO: 26 - RNA, 36 nt, Bluetongue virus
aucagcccggauagcauggcagcgacacuuuuuaac

SEQ ID NO: 27 - RNA, 41 nt, Bluetongue virus
guaaguguguagcgccgcauacccucccccguuagacagca

SEQ ID NO: 28 - RNA, 39 nt, Bluetongue virus
ccucggggcgccacucuaccuacugaucuuagguuaaug

SEQ ID NO: 29 - RNA, 35 nt, Bluetongue virus
uuagguuaaugguaauucgaaaccaucuagcggga

SEQ ID NO: 30 - RNA, 35 nt, Bluetongue virus
aauuugcugguucaagcuucucucgcuuuuugcgc

SEQ ID NO: 31 - RNA, 34 nt, Bluetongue virus
guaggagucugcaucgugagaucaaccacucuac

SEQ ID NO: 32 - RNA, 30 nt, Bluetongue virus
ugcuauuaccaugcuacagauguaagugau

SEQ ID NO: 33 - DNA, 35 bp, Bluetongue virus
ttaacctaagatcagtaggtagagtggcgccccga

SEQ ID NO: 34 - DNA, 35 bp, Bluetongue virus
ttaacctaaggacagtaggtagagtggcgccccaa

SEQ ID NO: 35 - DNA, 32 bp, Bluetongue virus
caaaccaagacagaggagagggcgccccgagg

SEQ ID NO: 36 - RNA, 39 nt, Bluetongue virus
cauuaaccuaagaucaguagguagaguggcgccccgagg

SEQ ID NO: 37 - RNA, 34 nt, Bluetongue virus
guagagugguugaucucacgaugcagacuccuac

SEQ ID NO: 38 - RNA, 39 nt, Bluetongue virus; identical to SEQ ID NO: 36

SEQ ID NO: 39 - RNA, 40 nt, Bluetongue virus
aucgugguagagugguugaucucacgaugcagacuccuac